(19) United States
(12) Patent Application Publication
(10) Pub. No.: US 2026/0088129 A1
(43) Pub. Date: Mar. 26, 2026

(54) METHODS AND SYSTEMS FOR DETERMINING VARIANT PROPERTIES USING MACHINE LEARNING

(71) Applicant: Foundation Medicine, Inc., Boston, MA (US)

(72) Inventors: Garrett M. FRAMPTON, Somerville, MA (US); Zheng KUANG, Boston, MA (US); Dean PAVLICK, Cambridge, MA (US)

(73) Assignee: Foundation Medicine, Inc., Boston, MA (US)

(21) Appl. No.: 19/112,148

(22) PCT Filed: Sep. 19, 2023
(86) PCT No.: PCT/US2023/074570

§ 371 Date: Mar. 14, 2025

Related U.S. Application Data
(60) Provisional application No. 63/408,237, filed on Sep. 20, 2022.

(51) Int. Cl. G16B 30/10; C12Q 1/6874; C12Q 1/6886; G16B 40/20

(52) U.S. Cl. CPC G16B 30/10; C12Q 1/6874; C12Q 1/6886; G16B 40/20; C12Q 2600/156

(57) ABSTRACT

Methods for determining a functional status of a variant are described. The methods may comprise, for example, receiving sequence read data associated with a plurality of samples; determining one or more feature attributes based on the sequence read data of the plurality of samples, the one or more feature attributes associated with one or more input features; categorizing the one or more feature attributes into a plurality of input feature categories based on the one or more feature attributes; obtaining one or more input feature values based on the plurality of input feature categories; inputting the one or more input feature values into a statistical model; and determining the functional status of the variant based on an output of the statistical model, wherein the functional status is indicative of a level of pathogenicity of the variant.

### CROSS REFERENCE TO RELATED APPLICATION

This application claims the benefit of U.S. Provisional Application No. 63/408,237, filed Sep. 20, 2022, the contents of which are hereby incorporated by reference in its entirety.

### FIELD OF THE INVENTION

The present disclosure relates generally to methods and systems for analyzing genomic profiling data, and more specifically to methods and systems for determining variant properties (e.g., functional status and origin) using genomic profiling data.

### BACKGROUND

Genomic sequencing enables the ability to identify genomic variants in a sample from an individual. Efforts have been made to classify genomic variants based on a level of pathogenicity, e.g., a known pathogenic variant, a likely pathogenic variant, a variant of unknown significance (VUS), or a benign variant. Variants can also be classified based on their origin, e.g., whether the variant is tumor derived, germline, or clonal hematopoiesis (CH) derived. These classifications may be used by healthcare providers to make treatment decisions. For example, if an individual's sample includes one or more pathogenic genomic variants, the healthcare provider may base a treatment decision on the therapies available for disease associated with those pathogenic genomic variants. For example, a patient with ovarian cancer with a BRCA2 alteration may be considered for PARP inhibitors. As another example, if a genomic variant in an individual's sample is determined to be a benign germline variant (e.g., non-tumor derived), the healthcare provider may decide that no treatment is necessary, because the variant is not an oncogenic driver of disease. Thus, accurately classifying genomic variants may allow healthcare providers to provide improved treatment options for patients to improve patient outcomes.

A number of genomic variants have been classified in the scientific literature. However, many genomic variants detected in samples from individuals suspected of having a disease (e.g., cancer) are classified as VUSs and have not been characterized in the literature as being associated with a particular treatment, despite these VUSs likely being driver mutations of disease. Conversely, some variants (particularly variants that occur infrequently in the population or in underrepresented populations) may be erroneously reported as functional, while these variants may actually be benign. Incorrect reporting of functional status confounds determinations of patient-specific pathogenic mechanisms and may lead to subpar clinical care.

As another example, understanding the origin of an alteration can have downstream consequences. For example, variants causing germline predispositions can be distinguished from variants that drive disease (e.g., cancer) in an individual. Disease-driving genomic variants may be detected both in the tumor DNA (e.g., tumor-derived) and in blood progenitor DNA (CH-derived). For example, certain alterations may be very rare, but such alterations may be prevalent in familial medical histories (e.g., BRCA2 chr13:32910462T>A has an allele frequency of less than 0.01%, but is seen in familial breast and ovarian cancer. Assuming that all drivers come from tumor DNA confounds the understanding of patient-specific pathogenic mechanisms in the tumor and may lead to subpar clinical care based on the molecular diagnosis.

Efforts to identify variant properties (e.g., functional status or somatic/germline origin) suffer from a number of deficiencies. For example, current methods to determine the functional status of variants (e.g., gene expression assays, protein functional assays, or cell phenotype assays) are typically low-throughput, constrained by data availability, and the scientific literature often does not include information regarding rare variants in less-studied genes. Large genomic databases may be used to empirically deduce the functional status of certain variants, typically be evaluating a rate of recurrence in cancer, identifying over-representation in certain cancer types, and identifying distributions across different ancestral groups. But these large databases may not include information regarding rare variants in less-studied genes. Further, the databases may disagree on the functional status for alterations that are now well-studied. Additionally, some methods rely on a manual process to evaluate and determine the functional status, which can be time consuming and costly. As another example, determining the CH status is typically performed using paired normal sequencing, which becomes cost prohibitive when performed at scale. Accordingly, there is a need to provide a method to accurately, efficiently, and cost effectively determine variant properties.

### BRIEF SUMMARY OF THE INVENTION

Embodiments of the present disclosure provide systems and methods to accurately, efficiently, and cost effectively determine genomic variant properties (e.g., functional status or origin) by analyzing a plurality of variants en masse based on sequence read data derived from a plurality of samples. In one or more examples, the disclosed methods and system use machine learning to classify currently unknown variants (e.g., VUSs) and can help to clarify (e.g., substantiate or refute) variants that are currently classified in scientific literature.

Accordingly, embodiments of the present disclosure can improve upon previous methods by permitting an investigation of the functional status of a plurality of variants (including rare and non-coding variants) at scale to classify the properties of the variants and identify one or more features that are associated with a particular functional status of a variant. In contrast, based on current methods an experienced pathologist would have to individually analyze tissue samples comprising genomic variants to recognize patterns of occurrence associated with a pathogenic nature, which can be very time consuming and particularly ineffective in the case of rare variants.

Embodiments of the present disclosure provide systems and methods for determining a functional status of one or more variants based on a plurality of samples. Methods according to the present disclosure comprise: providing a plurality of nucleic acid molecules obtained from a plurality of samples from a plurality of subjects; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads associated with the plurality of samples; determining, using the one or more processors, one or more feature attributes based on the sequence read data of the plurality of samples, the one or more feature attributes associated with one or more input features; categorizing, using the one or more processors, the one or more feature attributes into a plurality of input feature categories based on the one or more feature attributes, wherein an input feature of the one or more input features is associated with one or more input feature categories of the plurality of input feature categories; obtaining, using the one or more processors, one or more input feature values based on the plurality of input feature categories; inputting, using the one or more processors, the one or more input feature values into a statistical model; and determining, using the one or more processors, the functional status of a variant based on an output of the statistical model.

In one or more examples of embodiments of this disclosure, the functional status is indicative of a level of pathogenicity of the variant. In one or more examples of embodiments of this disclosure, the non-known pathogenic status comprises a likely pathogenic status of the variant, a variant of unknown significance status of the variant, a benign status of the variant, or a combination thereof. In one or more examples of embodiments of this disclosure, the plurality of input feature categories correspond to pre-determined categories. In one or more examples of embodiments of this disclosure, the method further comprises organizing a feature attribute of the one or more feature attributes into a first category based on a first input feature if a number of samples associated with the first input feature is below a pre-determined threshold.

In one or more examples of embodiments of this disclosure, the one or more input features are associated with one or more variant features, one or more sample features, one or more clinical features, or a combination thereof. In one or more examples of embodiments of this disclosure, the output of the statistical model is indicative of a functional status of the variant. In one or more examples of embodiments of this disclosure, the output of the statistical model comprises one or more feature scores indicative of a relative importance of the one or more input features.

In one or more examples of embodiments of this disclosure, the statistical model is a trained machine learning model and trained by: receiving, using the one or more processors, training data including one or more training feature values based on one or more training input feature categories associated with a plurality of training samples; and training, using the one or more processors, the machine learning model based on the training data. In one or more examples of embodiments of this disclosure, the one or more training feature categories are associated with one or more training features, the one or more training features comprising: one or more variant features, one or more sample features, one or more demographic features, or a combination thereof.

In one or more examples of embodiments of this disclosure, the training further comprise obtaining training data, comprising: receiving, using one or more processors, training sequence read data associated with the plurality of training samples; determining, using the one or more processors, one or more training feature attributes based on the training sequence read data; organizing, using the one or more processors, the one or more training feature attributes into the one or more training feature categories; obtaining the one or more training feature values based on the one or more training feature categories; inputting, using the one or more processors, the one or more training feature values into an untrained statistical model; predicting, using the one or more processors, the functional status of the variant based on the one or more training feature values; obtaining one or more training feature scores indicative of a relative importance of the one or more training features; updating one or more weights associated with a trained statistical model based on the one or more training feature scores. In one or more examples of the embodiment described above, the method further comprises organizing a training feature attribute of the one or more training feature attributes into a first category based on a first training feature, if a number of training samples of the plurality of training samples associated with the first training feature is below a pre-determined threshold.

In one or more examples of the embodiments described above, the subject is suspected of having or is determined to have cancer. In one or more examples of the embodiment described above, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In one or more examples of the embodiment described above, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In one or more examples of the embodiments described above, the method further comprises treating the subject with an anti-cancer therapy. In one or more examples of the embodiments described above, the anti-cancer therapy comprises a targeted anti-cancer therapy. In one or more examples of the embodiments described above, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In one or more examples of the embodiments described above, the method further comprises obtaining the plurality of samples from the plurality of subjects. In one or more examples of the embodiments described above, a sample of the plurality of samples comprises a tissue biopsy sample, a liquid biopsy sample, or a normal control. In one or more examples of the embodiments described above, the sample is a liquid biopsy sample and comprises blood, plasma, cerebrospinal fluid, sputum, stool, urine, or saliva. In one or more examples of the embodiments described above, the sample is a liquid biopsy sample and comprises circulating tumor cells (CTCs). In one or more examples of the embodiments described above, the sample is a liquid biopsy sample and comprises cell-free DNA (cfDNA), circulating tumor DNA (ctDNA), or any combination thereof.

In one or more examples of the embodiments described above, the plurality of nucleic acid molecules comprises a mixture of tumor nucleic acid molecules and non-tumor nucleic acid molecules. In one or more examples of the embodiment described above, the tumor nucleic acid molecules are derived from a tumor portion of a heterogeneous tissue biopsy sample, and the non-tumor nucleic acid molecules are derived from a normal portion of the heterogeneous tissue biopsy sample. In one or more examples of the embodiments described above, a sample of the plurality of samples comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In one or more examples of the embodiments described above, the one or more adapters comprise amplification primers, flow cell adaptor sequences, substrate adapter sequences, or sample index sequences. In one or more examples of the embodiments described above, the captured nucleic acid molecules are captured from the amplified nucleic acid molecules by hybridization to one or more bait molecules. In one or more examples of the embodiments described above, the one or more bait molecules comprise one or more nucleic acid molecules, each comprising a region that is complementary to a region of a captured nucleic acid molecule.

In one or more examples of the embodiments described above, amplifying nucleic acid molecules comprises performing a polymerase chain reaction (PCR) amplification technique, a non-PCR amplification technique, or an isothermal amplification technique. In one or more examples of the embodiments described above, the sequencing comprises use of a massively parallel sequencing (MPS) technique, whole genome sequencing (WGS), whole exome sequencing, targeted sequencing, direct sequencing, or Sanger sequencing technique. In such embodiments, the sequencing comprises massively parallel sequencing, and the massively parallel sequencing technique comprises next generation sequencing (NGS).

In one or more examples of the embodiments described above, the sequencer comprises a next generation sequencer. In one or more examples of the embodiments described above, one or more of the plurality of sequencing reads overlap one or more gene loci within one or more subgenomic intervals in a sample of the plurality of samples. In such embodiments the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more examples of the embodiments described above, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In one or more examples of the embodiments described above, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In one or more examples of the embodiments described above, the method further comprises generating, by the one or more processors, a report indicating the functional status of the variant. In such embodiments, the method further comprises transmitting the report to a healthcare provider. In such embodiments, the report is transmitted via a computer network or a peer-to-peer connection.

Embodiments of the present disclosure further provide methods for determining a functional status of a variant. These methods can comprise: receiving, using one or more processors, sequence read data associated with a plurality of samples; determining, using the one or more processors, one or more feature attributes based on the sequence read data of the plurality of samples, the one or more feature attributes associated with one or more input features; categorizing, using the one or more processors, the one or more feature attributes into a plurality of input feature categories based on the one or more feature attributes, wherein an input feature of the one or more input features is associated with one or more input feature categories of the plurality of input feature categories; obtaining, using the one or more processors, one or more input feature values based on the plurality of input feature categories; inputting, using the one or more processors, the one or more input feature values into a statistical model; and determining, using the one or more processors, the functional status of the variant based on an output of the statistical model, wherein the functional status is indicative of a level of pathogenicity of the variant.

In one or more examples of the embodiments described above, the functional status comprises a known pathogenic status of the variant or a non-known pathogenic status of the variant. In one or more examples of the embodiments described above, the non-known pathogenic status comprises a likely pathogenic status of the variant, a variant of unknown significance status of the variant, a benign status of the variant, or a combination thereof. In one or more examples of the embodiments described above, the non-known pathogenic status comprises a variant of unknown significance status of the variant, a benign status of the variant, or a combination thereof.

In one or more examples of the embodiments described above, the plurality of input feature categories correspond to pre-determined categories. In such embodiments, the pre-determined categories comprise quantiles based on the one or more input features.

In one or more examples of the embodiments described above, the method further comprises: inputting, using the one or more processors, the one or more feature attributes into a genomic database; determining, using the one or more processors, a gene co-mutation value indicative of a prevalence of gene co-mutations associated with the variant based on the genomic database; and inputting, using the one or more processors, the gene co-mutation value into the statistical model, wherein determining the functional status is based on the gene co-mutation value.

In one or more examples of the embodiments described above, the method further comprises organizing a feature attribute of the one or more feature attributes into a first category based on a first input feature if a number of samples associated with the first input feature is below a pre-determined threshold.

In one or more examples of the embodiments described above, the one or more input features are associated with one or more variant features, one or more sample features, one or more clinical features, or a combination thereof. In one or more examples of the embodiments described above, the method further comprises obtaining clinical information, wherein determining the one or more input features is further based on the clinical information. In one or more embodiments, the one or more variant features comprise a presence of a short variant, an absence of a short variant, a variant minor allele frequency, a germline status, a somatic status, a zygosity determination, a copy number alteration, a genomic rearrangement, or a combination thereof. In one or more examples of the embodiments described above, the one or more sample features comprise a bait-set, a tumor purity, a loss of heterozygosity (LOH) status, a LOH ploidy status, a LOH TP53 status, a LOH QC status, a microsatellite instability, a tumor mutational burden, a mutational signature, or a combination thereof. In one or more examples of the embodiments described above, the one or more clinical features comprise an age of the individual, a sex of the individual, a disease ontology, a genomic ancestry of the individual, or a combination thereof.

In one or more examples of the embodiments described above, the output of the statistical model is indicative of a functional status of the variant. In one or more examples of the embodiments described above, the output of the statistical model comprises one or more feature scores indicative of a relative importance of the one or more input features.

In one or more examples of the embodiments described above, the statistical model is a trained machine learning model and trained by: receiving, using the one or more processors, training data including one or more training feature values based on one or more training input feature categories associated with a plurality of training samples; and training, using the one or more processors, the machine learning model based on the training data. In such examples, the one or more training feature categories are associated with one or more training features, the one or more training features comprising: one or more variant features, one or more sample features, one or more demographic features, or a combination thereof.

In one or more examples of the embodiments described above, the method further comprises obtaining training data, comprising: receiving, using one or more processors, training sequence read data associated with the plurality of training samples; determining, using the one or more processors, one or more training feature attributes based on the training sequence read data; organizing, using the one or more processors, the one or more training feature attributes into the one or more training feature categories; obtaining the one or more training feature values based on the one or more training feature categories; inputting, using the one or more processors, the one or more training feature values into an untrained statistical model; predicting, using the one or more processors, the functional status of the variant based on the one or more training feature values; obtaining one or more training feature scores indicative of a relative importance of the one or more training features; updating one or more weights associated with a trained statistical model based on the one or more training feature scores. In such examples, the method further comprises organizing a training feature attribute of the one or more training feature attributes into a first category based on a first training feature, if a number of training samples of the plurality of training samples associated with the first training feature is below a pre-determined threshold.

In one or more examples of the embodiments described above, the method further comprises: inputting, using the one or more processors, the one or more training feature attributes into a genomic database; determining, using the one or more processors, a gene co-mutation value indicative of a number of gene co-mutations associated with the variant based on the genomic database; and inputting, using the one or more processors, the gene co-mutation value into the untrained statistical model, wherein predicting the functional status is further based on the gene co-mutation value. In one or more examples of the embodiments described above, the method further comprises obtaining a pre-defined functional status of the variant based on an orthogonal method; labeling the variant based on the pre-defined functional status; and inputting the labeled pre-defined functional status of the variant into the untrained statistical model, wherein updating one or more weights is based on the labeled pre-defined functional status. In such embodiments, the orthogonal method is based on variants identified in literature, variants identified in catalogue of somatic mutations in cancer (COSMIC), or a combination thereof.

In one or more examples of the embodiments described above, the statistical model is a machine learning model. In one or more examples of the embodiments described above, the statistical model is part of a machine learning process. In one or more examples of the embodiments described above, the statistical model includes an artificial intelligence learning model. In one or more examples of the embodiments described above, the statistical model comprises a random forest model. In one or more examples of the embodiments described above, the statistical model comprises at least one of an extreme gradient boosting model, a logistic regression model, an elastic net model, a ridge regression model, a support vector machine model, a naïve-based model, a Gaussian naïve-based model, a limited expectation maximization model, a gradient boosting ensemble model, an adaboost model, a bagging model, a neural network model, a backpropagation model, a stochastic gradient descent model, a non-linear regression model, and a multivariate regression model.

In one or more examples of the embodiments described above, the method further comprises reclassifying, using the one or more processors, the variant based on the functional status. In one or more examples of the embodiments described above, the method further comprises generating, using the one or more processors, a report comprising the functional status of the variant. In one or more examples of the embodiments described above, the method further comprises assigning, using the one or more processors, a therapy for an individual based on the functional status. In one or more examples of the embodiments described above, the method further comprises determining, using the one or more processors, a treatment decision for an individual based on the functional status. In one or more examples of the embodiments described above, the method further comprises selecting an anti-cancer therapy to administer to the subject based on the determination of the functional status. In one or more examples of the embodiments described above, the method further comprises administering, using the one or more processors, a treatment to an individual based on the functional status. In one or more examples of the embodiments described above, the method further comprises determining an effective amount of an anti-cancer therapy to administer to the subject based on the determination of the functional status. In such embodiments, the anti-cancer therapy comprises chemotherapy, radiation therapy, immunotherapy, a targeted therapy, or surgery. In one or more examples of the embodiments described above, the method further comprises monitoring, using the one or more processors, a prognosis of an individual based on the functional status. In one or more examples of the embodiments described above, the method further comprises predicting, using the one or more processors, one or more clinical outcomes based on the functional status.

In one or more examples of the embodiments described above, the sequence read data is based on whole genome sequencing, targeted exome sequencing, or a combination thereof. In one or more examples of the embodiments described above, the sequence read data is derived from a tissue biopsy sample, a liquid biopsy sample, or a combination thereof.

Embodiments of the present disclosure further provide methods for diagnosing a disease, the method comprising: diagnosing that a subject has the disease based on a determination of the functional status for a variant based on a plurality of samples, wherein the functional status is determined according to the methods described above. Embodiments of the present disclosure further provide methods of selecting an anti-cancer therapy, the method comprising: responsive to determining a functional status for a variant based on a plurality of samples, selecting an anti-cancer therapy for the subject, wherein the functional status is determined according to the methods described above. Embodiments of the present disclosure further provide methods of treating a cancer in a subject, comprising: responsive to determining a functional status for a variant based on a plurality of samples, administering an effective amount of an anti-cancer therapy to the subject, wherein the functional status is determined according to the methods described above.

Embodiments of the present disclosure further provide methods for monitoring cancer progression or recurrence in a subject, the method comprising: determining a presence of one or more first variants in a first sample obtained from the subject at a first time point, determining respective functional statuses of the one or more first variants, wherein the respective functional statuses are based on the functional status of the variant determined according to the methods described above; determining a presence of one or more second variants in a second sample obtained from the subject at a second time point; determining respective functional statuses of the one or more second variants, and comparing the one or more first variants and the one or more second variants in view of the respective functional statues, thereby monitoring the cancer progression or recurrence. In such embodiments, the respective functional statuses for the second sample is determined according to the methods described above. In one or more embodiments, the method further comprises selecting an anti-cancer therapy for the subject in response to the cancer progression. In one or more embodiments, the method further comprises administering an anti-cancer therapy to the subject in response to the cancer progression.

In one or more embodiments, the method further comprises adjusting an anti-cancer therapy for the subject in response to the cancer progression. In one or more embodiments, the method further comprises adjusting a dosage of the anti-cancer therapy or selecting a different anti-cancer therapy in response to the cancer progression. In such embodiments, the method further comprises administering the adjusted anti-cancer therapy to the subject.

In one or more embodiments, the first time point is before the subject has been administered an anti-cancer therapy, and wherein the second time point is after the subject has been administered the anti-cancer therapy. In one or more embodiments, the subject has a cancer, is at risk of having a cancer, is being routine tested for cancer, or is suspected of having a cancer. In one or more embodiments, the cancer is a solid tumor. In one or more embodiments, the cancer is a hematological cancer. In one or more embodiments, the anti-cancer therapy comprises chemotherapy, radiation therapy, immunotherapy, a targeted therapy, or surgery.

In one or more embodiments of the methods described above, the method further comprises determining, identifying, or applying the functional status as a diagnostic metric associated with a sample of the plurality of samples.

In one or more embodiments of the methods described above, the method further comprises generating a genomic profile for the subject based on the determination of the functional status. In such embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In one or more embodiments, the genomic profile for the subject further comprises results from a nucleic acid sequencing-based test. In one or more embodiments, the method further comprises selecting an anti-cancer therapy, administering an anti-cancer therapy, or applying an anti-cancer therapy to the subject based on the generated genomic profile.

According to one or more methods of the present disclosure, the determination of the functional status of the variant based on a plurality of samples is used in making suggested treatment decisions for the subject. According to one or more methods of the present disclosure, the determination of the functional status of the variant based on a plurality of samples for the sample is used in applying or administering a treatment to the subject.

Systems in accordance with embodiments of the present disclosure may include: one or more processors and a memory communicatively coupled to the one or more processors. The memory may be configured to store instructions that, when executed by the one or more processors, cause the system to perform a method comprising: receiving, at the one or more processors, sequence read data associated with a plurality of samples; determining, using the one or more processors, one or more feature attributes based on the sequence read data of the plurality of samples, the one or more feature attributes associated with one or more input features; categorizing, using the one or more processors, the one or more feature attributes into a plurality of input feature categories based on the one or more feature attributes, wherein an input feature of the one or more input features is associated with one or more input feature categories of the plurality of input feature categories; obtaining, using the one or more processors, one or more input feature values based on the plurality of input feature categories; inputting, using the one or more processors, the one or more input feature values into a statistical model; and determining, using the one or more processors, a functional status of a variant based on an output of the statistical model.

Embodiments of the present disclosure further provide non-transitory computer-readable storage mediums. Non-transitory computer-readable storage mediums can be configured to store one or more programs, the one or more programs comprising instructions, which when executed by one or more processors of a system, cause the system to: receive sequence read data associated with a plurality of samples; determine one or more feature attributes based on the sequence read data of the plurality of samples, the one or more feature attributes associated with one or more input features; categorize the one or more feature attributes into a plurality of input feature categories based on the one or more feature attributes, wherein an input feature of the one or more input features is associated with one or more input feature categories of the plurality of input feature categories; obtain one or more input feature values based on the plurality of input feature categories; input the one or more input feature values into a statistical model; and determine a functional status of a variant based on an output of the statistical model.

Embodiments of the present disclosure further include methods for determining an origin of a variant based on a plurality of samples. The origin of a variant may refer to whether the variant is tumor derived or non-tumor derived (e.g., germline or clonal hematopoiesis (CH) derived). Methods for determining the origin of a variant can comprise: receiving, using one or more processors, sequence read data associated with a plurality of samples; determining, using the one or more processors, one or more input feature values based on the sequence read data of the plurality of samples; inputting, using the one or more processors, the one or more input feature values into a statistical mod receiving, using one or more processors, sequence read data associated with a plurality of samples; determining, using the one or more processors, one or more feature attributes based on the sequence read data of the plurality of samples, the one or more feature attributes associated with one or more input features; organizing, using the one or more processors, the one or more feature attributes into a plurality of input feature categories based on the one or more feature attributes, wherein an input feature of the one or more input features is associated with one or more input feature categories of the plurality of input feature categories; obtaining, using the one or more processors, one or more input feature values based on the plurality of input feature categories; inputting, using the one or more processors, the one or more input feature values into a statistical model; and determining, using the one or more processors, the origin of the variant based on an output of the statistical model.

In one or more embodiments of the method described above, the origin of the variant comprises one of clonal hematopoiesis (CH) derived, tumor derived, or germline.

In one or more examples of the embodiments described above, the plurality of input feature categories correspond to pre-determined categories. In such embodiments, the pre-determined categories comprise quantiles based on the one or more input features.

In one or more examples of the embodiments described above, the method further comprises: inputting, using the one or more processors, the one or more feature attributes into a genomic database; determining, using the one or more processors, a gene co-mutation value indicative of a prevalence of gene co-mutations associated with the variant based on the genomic database; and inputting, using the one or more processors, the gene co-mutation value into the statistical model, wherein determining the origin is based on the gene co-mutation value.

In one or more examples of the embodiments described above, the method further comprises organizing a feature attribute of the one or more feature attributes into a first category based on a first input feature if a number of samples associated with the first input feature is below a pre-determined threshold.

In one or more examples of the embodiments described above, the one or more input features are associated with one or more variant features, one or more sample features, one or more clinical features, or a combination thereof. In one or more examples of the embodiments described above, the method further comprises obtaining clinical information, wherein determining the one or more input features is further based on the clinical information. In one or more embodiments, the one or more variant features comprise a presence of a short variant, an absence of a short variant, a variant minor allele frequency, a germline status, a somatic status, a zygosity determination, a copy number alteration, a genomic rearrangement, or a combination thereof. In one or more examples of the embodiments described above, the one or more sample features comprise a bait-set, a tumor purity, a loss of heterozygosity (LOH) status, a LOH ploidy status, a LOH TP53 status, a LOH QC status, a microsatellite instability, a tumor mutational burden, a mutational signature, or a combination thereof. In one or more examples of the embodiments described above, the one or more clinical features comprise an age of the individual, a sex of the individual, a disease ontology, a genomic ancestry of the individual, or a combination thereof.

In one or more examples of the embodiments described above, the output of the statistical model is indicative of the origin of the variant. In one or more examples of the embodiments described above, the output of the statistical model comprises one or more feature scores indicative of a relative importance of the one or more input features.

In one or more examples of the embodiments described above, the statistical model is a trained machine learning model and trained by: receiving, using the one or more processors, training data including one or more training feature values based on one or more training input feature categories associated with a plurality of training samples; and training, using the one or more processors, the machine learning model based on the training data. In such examples, the one or more training feature categories are associated with one or more training features, the one or more training features comprising: one or more variant features, one or more sample features, one or more demographic features, or a combination thereof.

In one or more examples of the embodiments described above, the method further comprises obtaining training data, comprising: receiving, using one or more processors, training sequence read data associated with the plurality of training samples; determining, using the one or more processors, one or more training feature attributes based on the training sequence read data; organizing, using the one or more processors, the one or more training feature attributes into the one or more training feature categories; obtaining the one or more training feature values based on the one or more training feature categories; inputting, using the one or more processors, the one or more training feature values into an untrained statistical model; predicting, using the one or more processors, the origin of the variant based on the one or more training feature values; obtaining one or more training feature scores indicative of a relative importance of the one or more training features; updating one or more weights associated with a trained statistical model based on the one or more training feature scores. In such examples, the method further comprises organizing a training feature attribute of the one or more training feature attributes into a first category based on a first training feature, if a number of training samples of the plurality of training samples associated with the first training feature is below a pre-determined threshold.

In one or more examples of the embodiments described above, the method further comprises: inputting, using the one or more processors, the one or more training feature attributes into a genomic database; determining, using the one or more processors, a gene co-mutation value indicative of a number of gene co-mutations associated with the variant based on the genomic database; and inputting, using the one or more processors, the gene co-mutation value into the untrained statistical model, wherein predicting the origin is further based on the gene co-mutation value. In one or more examples of the embodiments described above, the method further comprises obtaining a pre-defined origin of the variant based on an orthogonal method; labeling the variant based on the pre-defined origin; and inputting the labeled pre-defined origin of the variant into the untrained statistical model, wherein updating one or more weights is based on the labeled pre-defined origin. In such embodiments, the orthogonal method is based on variants identified in literature, variants identified in catalogue of somatic mutations in cancer (COSMIC), or a combination thereof.

In one or more examples of the embodiments described above, the statistical model is a machine learning model. In one or more examples of the embodiments described above, the statistical model is part of a machine learning process. In one or more examples of the embodiments described above, the statistical model includes an artificial intelligence learning model. In one or more examples of the embodiments described above, the statistical model comprises a random forest model. In one or more examples of the embodiments described above, the statistical model comprises at least one of an extreme gradient boosting model, a logistic regression model, an elastic net model, a ridge regression model, a support vector machine model, a naïve-based model, a Gaussian naïve-based model, a limited expectation maximization model, a gradient boosting ensemble model, an adaboost model, a bagging model, a neural network model, a backpropagation model, a stochastic gradient descent model, a non-linear regression model, and a multivariate regression model.

In one or more examples of the embodiments described above, the method further comprises reclassifying, using the one or more processors, the variant based on the origin. In one or more examples of the embodiments described above, the method further comprises generating, using the one or more processors, a report comprising the origin of the variant. In one or more examples of the embodiments described above, the method further comprises assigning, using the one or more processors, a therapy for an individual based on the origin. In one or more examples of the embodiments described above, the method further comprises determining, using the one or more processors, a treatment decision for an individual based on the origin. In one or more examples of the embodiments described above, the method further comprises selecting an anti-cancer therapy to administer to the subject based on the determination of the origin. In one or more examples of the embodiments described above, the method further comprises administering, using the one or more processors, a treatment to an individual based on the origin. In one or more examples of the embodiments described above, the method further comprises determining an effective amount of an anti-cancer therapy to administer to the subject based on the determination of the origin. In such embodiments, the anti-cancer therapy comprises chemotherapy, radiation therapy, immunotherapy, a targeted therapy, or surgery. In one or more examples of the embodiments described above, the method further comprises monitoring, using the one or more processors, a prognosis of an individual based on the origin. In one or more examples of the embodiments described above, the method further comprises predicting, using the one or more processors, one or more clinical outcomes based on the origin.

In one or more examples of the embodiments described above, the sequence read data is based on whole genome sequencing, targeted exome sequencing, or a combination thereof. In one or more examples of the embodiments described above, the sequence read data is derived from a tissue biopsy sample, a liquid biopsy sample, or a combination thereof.

Embodiments of the present disclosure further provide methods for diagnosing a disease, the method comprising: diagnosing that a subject has the disease based on a determination of the origin for a variant based on a plurality of samples, wherein the origin is determined according to the methods described above. Embodiments of the present disclosure further provide methods of selecting an anti-cancer therapy, the method comprising: responsive to determining an origin for a variant based on a plurality of samples, selecting an anti-cancer therapy for the subject, wherein the origin is determined according to the methods described above. Embodiments of the present disclosure further provide methods of treating a cancer in a subject, comprising: responsive to determining an origin for a variant based on a plurality of samples, administering an effective amount of an anti-cancer therapy to the subject, wherein the origin is determined according to the methods described above.

Embodiments of the present disclosure further provide methods for monitoring cancer progression or recurrence in a subject, the method comprising: determining a presence of one or more first variants in a first sample obtained from the subject at a first time point, determining respective origins of the one or more first variants, wherein the respective origins are based on the origin of the variant determined according to the methods described above; determining a presence of one or more second variants in a second sample obtained from the subject at a second time point; determining respective origins of the one or more second variants, and comparing the one or more first variants and the one or more second variants in view of the respective functional statues, thereby monitoring the cancer progression or recurrence. In such embodiments, the respective origins for the second sample is determined according to the methods described above. In one or more embodiments, the method further comprises selecting an anti-cancer therapy for the subject in response to the cancer progression. In one or more embodiments, the method further comprises administering an anti-cancer therapy to the subject in response to the cancer progression.

In one or more embodiments, the method further comprises adjusting an anti-cancer therapy for the subject in response to the cancer progression. In one or more embodiments, the method further comprises adjusting a dosage of the anti-cancer therapy or selecting a different anti-cancer therapy in response to the cancer progression. In such embodiments, the method further comprises administering the adjusted anti-cancer therapy to the subject.

In one or more embodiments, the first time point is before the subject has been administered an anti-cancer therapy, and wherein the second time point is after the subject has been administered the anti-cancer therapy. In one or more embodiments, the subject has a cancer, is at risk of having a cancer, is being routine tested for cancer, or is suspected of having a cancer. In one or more embodiments, the cancer is a solid tumor. In one or more embodiments, the cancer is a hematological cancer. In one or more embodiments, the anti-cancer therapy comprises chemotherapy, radiation therapy, immunotherapy, a targeted therapy, or surgery.

In one or more embodiments of the methods described above, the method further comprises determining, identifying, or applying the origin as a diagnostic metric associated with a sample of the plurality of samples.

In one or more embodiments of the methods described above, the method further comprises generating a genomic profile for the subject based on the determination of the origin of the variant. In such embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In one or more embodiments, the genomic profile for the subject further comprises results from a nucleic acid sequencing-based test. In one or more embodiments, the method further comprises selecting an anti-cancer therapy, administering an anti-cancer therapy, or applying an anti-cancer therapy to the subject based on the generated genomic profile.

According to one or more methods of the present disclosure, the determination of the origin of the variant based on a plurality of samples is used in making suggested treatment decisions for the subject. According to one or more methods of the present disclosure, the determination of the origin of the variant based on a plurality of samples for the sample is used in applying or administering a treatment to the subject.

Systems in accordance with embodiments of the present disclosure may include: one or more processors and a memory communicatively coupled to the one or more processors. The memory may be configured to store instructions that, when executed by the one or more processors, cause the system to perform a method comprising: receiving, at the one or more processors, sequence read data associated with a plurality of samples; determining, using the one or more processors, one or more feature attributes based on the sequence read data of the plurality of samples, the one or more feature attributes associated with one or more input features; categorizing, using the one or more processors, the one or more feature attributes into a plurality of input feature categories based on the one or more feature attributes, wherein an input feature of the one or more input features is associated with one or more input feature categories of the plurality of input feature categories; obtaining, using the one or more processors, one or more input feature values based on the plurality of input feature categories; inputting, using the one or more processors, the one or more input feature values into a statistical model; and determining, using the one or more processors, an origin of a variant based on an output of the statistical model.

Embodiments of the present disclosure further provide non-transitory computer-readable storage mediums. Non-transitory computer-readable storage mediums can be configured to store one or more programs, the one or more programs comprising instructions, which when executed by one or more processors of a system, cause the system to: receive sequence read data associated with a plurality of samples; determine one or more feature attributes associated with one or more input features based on the sequence read data of the plurality of samples; categorize the one or more feature attributes into a plurality of input feature categories, wherein an input feature of the one or more input features is associated with one or more input feature categories of the plurality of input feature categories; obtain one or more input feature values based on the plurality of input feature categories; input the one or more input feature values into a statistical model; and determine an origin of a variant based on an output of the statistical model.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process for determining a functional status of a variant, according to embodiments of the present disclosure.

FIG. 2A provides a non-limiting example of input feature types for a statistical model according to embodiments of the present disclosure.

FIG. 2B provides a non-limiting example of input feature types for a statistical model according to embodiments of the present disclosure.

FIG. 2C provides a non-limiting example of input feature types for a statistical model according to embodiments of the present disclosure.

FIG. 2D provides a non-limiting example of input feature types and respective feature attributes for exemplary samples according to embodiments of the present disclosure.

FIG. 3A provides a non-limiting example of an input feature for a statistical model according to embodiments of the present disclosure.

FIG. 3B provides a non-limiting example of an input feature for a statistical model according to embodiments of the present disclosure.

FIG. 4 provides a non-limiting example of a process for determining an origin of a variant, according to embodiments of the present disclosure.

FIG. 5 provides a non-limiting example of a process for determining a variant property of a variant, according to embodiments of the present disclosure.

FIG. 6 provides a non-limiting exemplary flow chart for training a model to determine a variant property, according to embodiments of the present disclosure.

FIG. 7 provides a non-limiting exemplary flow chart for training a model to determine a variant property of a variant, according to embodiments of the present disclosure.

FIG. 8 depicts an exemplary computing device or system in accordance with one embodiment of the present disclosure.

FIG. 9 depicts an exemplary computer system or computer network, in accordance with some instances of the systems described herein.

### DETAILED DESCRIPTION

Embodiments of the present disclosure provide systems and methods to accurately, efficiently, and cost effectively determine genomic variant properties (e.g., functional status or origin) by analyzing a plurality of variants en masse based on sequence read data derived from a plurality of samples. In one or more examples, the disclosed methods and system use machine learning to classify currently unknown variants (e.g., VUSs) and can help to clarify (e.g., substantiate or refute) variants that are currently classified in scientific literature.

Accordingly, embodiments of the present disclosure can improve upon previous methods by permitting an investigation of the functional status of a plurality of variants (including rare and non-coding variants) at scale to classify the properties of the variants and identify one or more features that are associated with a particular functional status of a variant. In contrast, based on current methods an experienced pathologist would have to individually analyze tissue samples comprising genomic variants to recognize patterns of occurrence associated with a pathogenic nature, which can be very time consuming and particularly ineffective in the case of rare variants.

Embodiments of the present disclosure further provide methods for determining a functional status of a variant. These methods can comprise: receiving, using one or more processors, sequence read data associated with a plurality of samples; determining, using the one or more processors, one or more feature attributes based on the sequence read data of the plurality of samples, the one or more feature attributes associated with one or more input features; categorizing, using the one or more processors, the one or more feature attributes into a plurality of input feature categories based on the one or more feature attributes, wherein an input feature of the one or more input features is associated with one or more input feature categories of the plurality of input feature categories; obtaining, using the one or more processors, one or more input feature values based on the plurality of input feature categories; inputting, using the one or more processors, the one or more input feature values into a statistical model; and determining, using the one or more processors, the functional status of the variant based on an output of the statistical model, wherein the functional status is indicative of a level of pathogenicity of the variant.

Embodiments of the present disclosure further include methods for determining an origin of a variant based on a plurality of samples. The origin of a variant may refer to whether the variant is tumor derived or non-tumor derived (e.g., germline or clonal hematopoiesis (CH) derived). Methods for determining the origin of a variant can comprise: receiving, using one or more processors, sequence read data associated with a plurality of samples; determining, using the one or more processors, one or more input feature values based on the sequence read data of the plurality of samples; inputting, using the one or more processors, the one or more input feature values into a statistical mod receiving, using one or more processors, sequence read data associated with a plurality of samples; determining, using the one or more processors, one or more feature attributes based on the sequence read data of the plurality of samples, the one or more feature attributes associated with one or more input features; organizing, using the one or more processors, the one or more feature attributes into a plurality of input feature categories based on the one or more feature attributes, wherein an input feature of the one or more input features is associated with one or more input feature categories of the plurality of input feature categories; obtaining, using the one or more processors, one or more input feature values based on the plurality of input feature categories; inputting, using the one or more processors, the one or more input feature values into a statistical model; and determining, using the one or more processors, the origin of the variant based on an output of the statistical model.

In one or more examples of the embodiments described above, the functional status comprises a known pathogenic status of the variant or a non-known pathogenic status of the variant. In one or more examples of the embodiments described above, the non-known pathogenic status comprises a likely pathogenic status of the variant, a variant of unknown significance status of the variant, a benign status of the variant, or a combination thereof. In one or more examples of the embodiments described above, the non-known pathogenic status comprises a variant of unknown significance status of the variant, a benign status of the variant, or a combination thereof. In one or more examples of the embodiments described above, the output of the statistical model is indicative of a functional status of the variant. In one or more examples of the embodiments described above, the output of the statistical model comprises one or more feature scores indicative of a relative importance of the one or more input features.

In one or more embodiments of the methods described above, the origin of the variant comprises one of clonal hematopoiesis (CH) derived, tumor derived, or germline. In one or more examples of the embodiments described above, the plurality of input feature categories correspond to pre-determined categories. In such embodiments, the pre-determined categories comprise quantiles based on the one or more input features. In one or more examples of the embodiments described above, the output of the statistical model is indicative of the origin of the variant. In one or more examples of the embodiments described above, the output of the statistical model comprises one or more feature scores indicative of a relative importance of the one or more input features.

In one or more examples of the embodiments described above, the method further comprises organizing a feature attribute of the one or more feature attributes into a first category based on a first input feature if a number of samples associated with the first input feature is below a pre-determined threshold. In one or more examples of the embodiments described above, the one or more input features are associated with one or more variant features, one or more sample features, one or more clinical features, or a combination thereof.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The term “known pathogenic variant” may refer to a variant that has a known and documented relevance to disease, e.g., recognized in the scientific literature as being an oncogenic driver of disease.

The term “likely pathogenic variant” may refer to a variant that is potentially relevant to pathogenicity, e.g., as supported by scientific literature, research, and or the genomic data from the one or more samples.

The term “variant of unknown significance” (VUS) may refer to a variant that does not have a well-known or documented association with disease, e.g., there may not be sufficient evidence to identify the variant as a known pathogenic or likely pathogenic variant.

The term “benign variant” may refer to a variant that is determined to not be relevant to disease.

The section headings used herein are for organizational purposes only and are not to be construed as limiting the subject matter described.

### Methods for Determining a Variant Property Based on a Plurality of Samples

Embodiments of the present disclosure provide systems and methods to accurately, efficiently, and cost effectively determine genomic variant properties (e.g., functional status or origin) by analyzing a plurality of variants en masse based on sequence read data derived from a plurality of samples. In one or more examples, the disclosed methods and system use machine learning to classify currently unknown variants (e.g., VUSs) and can help to clarify (e.g., substantiate or refute) variants that are currently classified in scientific literature.

This improves upon previous methods by permitting an investigation of the variant property of a plurality of variants (including rare and non-coding variants) at scale to classify the properties of the variants and identify one or more features that are associated with a particular status of a variant. In contrast, for example, based on current methods an experienced pathologist would have to individually analyze variants to recognize patterns of occurrences regarding a pathogenic nature, which can be very time consuming and ineffective, e.g., in the case of rare variants.

Embodiments of the present disclosure provide processes for classifying a plurality of genomic variants based on a variant property. As used herein, a variant property may refer to a functional status of a variant or an origin of a variant. As used herein, the functional status may refer to a level of pathogenicity of the variant. In one or more examples, the origin of a variant may refer to whether a variant is germline or originated from a tumor.

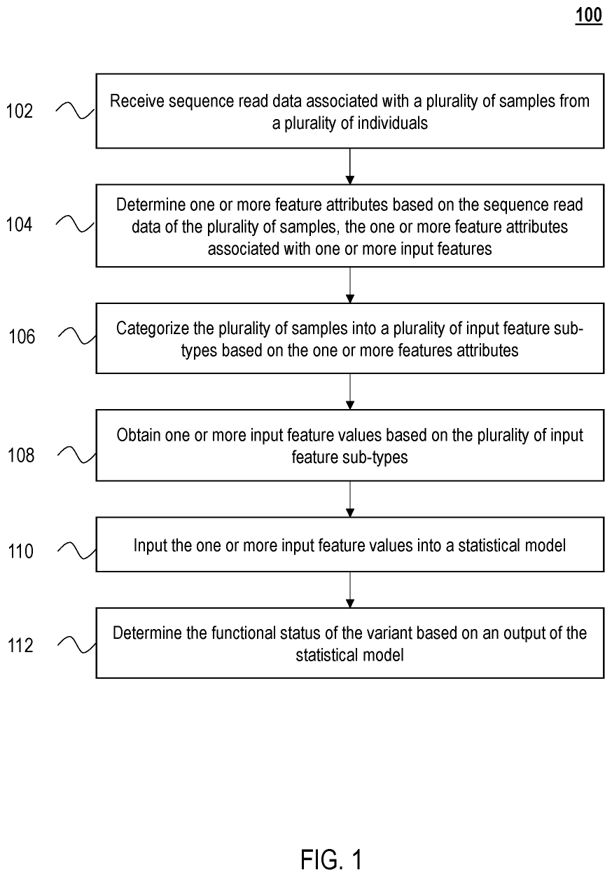

FIG. 1 provides a non-limiting example of a process 100 for determining the functional status of a variant based on a plurality of samples.

Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100.

Accordingly, the operations as illustrated (and described in greater detail below) are exemplary by nature and, as such, should not be viewed as limiting.

At block 102 of FIG. 1, the system can receive sequence read data associated with a plurality of samples from a plurality of individuals. In some instances, the sequence read data may be associated with one or more genomic variants in the samples. In one or more examples, the sequence read data may be derived from single region sequencing (e.g., sequencing of a single tissue biopsy sample collected from a tumor of an individual). In some instances, the genomic data comprising sequence read data may be derived from multi-region sequencing (e.g., sequencing of multiple tissue biopsy samples collected from a tumor of an individual). In some instances, the genomic data comprising sequence read data may be derived from single cell sequencing data as opposed to bulk tumor sequencing. In some instances, the genomic data comprising sequence read data may be derived from sequencing the circulating tumor DNA in a liquid biopsy sample.

In some instances, the genomic data comprising sequence read data may be derived from targeted sequencing, e.g., targeted exome sequencing. In some instances, the genomic data comprising sequence read data may be derived from, e.g., whole genome or whole exome sequencing, as opposed to targeted exome sequencing to increase the number of genomic features (e.g., the number of short variants) detected. In one or more examples, the sequence read data may be received by the system as a BAM file.

In one or more examples, the sequence read data may be indicative of one or more variant level or sample level features. The variant level features may correspond to features characterizing a particular variant (e.g., a TP53 variant). The Sample level features may correspond to features characterizing a particular sample (e.g., sample 0 from individual Y). For example, the sequence read data may be indicative of a presence or absence of one or more short variants (SVs) in the plurality of samples from the plurality of individuals individual. In one or more examples, the sequence read data may also be indicative of the presence or absence of genomic characteristics, such as copy number alterations, zygosity, minor allele frequency (MAF), rearrangements, insertions, deletions, fusions, chromosomal aneuploidy, whole genome doubling, short variant (SV) signatures, microsatellite instability (MSI) status, tumor mutational burden (TMB), loss of heterozygosity (LOH), or any combination thereof.

At block 104 of FIG. 1, the system can determine one or more feature attributes associated with one or more input features based on the sequence read data of the plurality of samples. In one or more examples, the input features may include variant level features (e.g., features characterizing a particular variant identified in a sample) sample level features (e.g., features characterizing a particular sample), and clinical features (e.g., features associated with the individual, disease, and/or diagnostic test). In one or more examples, the clinical features are determined based on non-genomic sequence read data. In one or more examples, additional information obtained via an individual's medical health records and/or test requisition may be used to determine the one or more feature attributes. In one or more examples, the feature attributes may correspond to a value and/or category associated with an input feature (e.g., input feature type). Exemplary feature attributes and input features will be described in greater detail below.

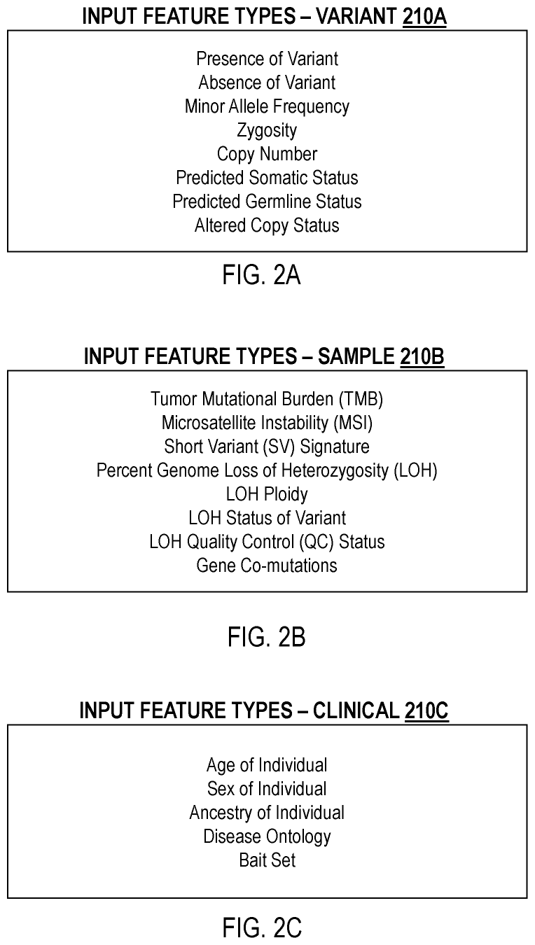

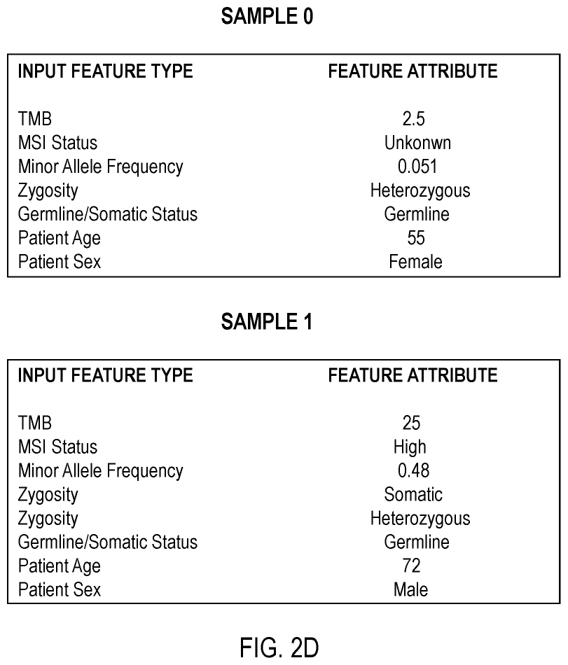

FIG. 2A illustrates exemplary variant input feature types 210A. As shown in the figure, the variant level input feature types can include one or more of a presence of a variant, an absence of a variant, a minor allele frequency (MAF) of a variant, a zygosity, a copy number, a predicted somatic/germline status, and/or an altered copy status.

For example, the system may determine a presence and/or absence of a genetic variant (e.g., TP53) for the plurality of samples. In one or more examples, the system can determine a feature attribute corresponding to an MAF value of the genetic variant (e.g., a TP53 variant) identified in the plurality of samples. In one or more examples, the system can determine a feature attribute corresponding to a zygosity category and/or a copy number category of the genetic variants (e.g., a TP53 variant) identified in the plurality of samples. In one or more examples, exemplary zygosity categories may include a heterogeneous call, a homozygous call, a not in tumor call, and/or an unknown call. In some instances, not in tumor may indicate that the variant copy is not seen in the tumor, so there may be no zygosity in the tumor to mark and/or there may be a benign germline withstanding copy number loss in tumor. In another instance, not in tumor may indicate a zygosity call was no attempted. In one or more embodiments, the system can determine a feature attribute corresponding to a somatic category and/or a germline category indicative of whether the genetic variant (e.g., a TP53 variant) is derived from somatic or germline cells. In one or more examples, exemplary somatic/germline categories may include an ambiguous copy number alteration model call, an ambiguous germline and somatic call, an ambiguous neither germline nor somatic call, a germline call, a probable germline call, a somatic call, a probable somatic call, a sub-clonal somatic call, an insufficient tumor purity call (e.g., tumor purity less than 95%), a missing segment call, and/or an unknown call. In one or more examples, the system can determine a feature attribute corresponding to an altered copy value associated with the genetic variant (e.g., a TP53 variant). An altered copy value may correspond to a number of copies altered for that variant. For example, in a triploid tumor with one altered copy, the zygosity call would be heterozygous and the altered copy value would be one. While these determinations are described with respect to a particular variant, e.g., a TP53 variant, a skilled artisan would understand that the system may perform these determinations with respect to a plurality of variants, (e.g., ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof).

FIG. 2B illustrates exemplary sample input feature types 210B. As shown in the figure, the sample level input features can include one or more of a tumor mutational burden (TMB), a microsatellite instability (MSI), a short variant (SV) signature, a sample-wide percent genome loss of heterozygosity (LOH), a genome-wide LOH ploidy, a sample-wide LOH status of a variant, a LOH quality control (QC) status, and/or gene co-mutations.

For example, the system can determine a feature attribute corresponding to a TMB value of the sample. As another example, the system can determine a feature attribute corresponding to a microsatellite instability (MSI) category of the sample. In one or more examples, the MSI categories can include, but are not limited to, an unknown MSI, a high MSI, an MSI stable status and the like. In one or more examples, the system can determine a feature attribute corresponding to one or more short variant signature categories. In one or more examples, the short variant signature categories can include, but are not limited to, MMR, APOBEC, Tobacco, UV, Alkylating, POLE, etc. In one or more examples, the system can determine a feature attribute corresponding to a sample-wide percent genome loss of heterozygosity (LOH) value, where the value is predicted based on copy number modeling.

In one or more examples, the system can determine a feature attribute corresponding to a ploidy of genome-wide LOH value. In one or more examples, the system can determine a feature attribute corresponding to a sample-wide LOH status of a variant category, indicative of the LOH status for a particular variant. For example, if the variant is a TP53 variant, the sample-wide LOH status categories may include, but are not limited to, whether the sample has TP53 LOH, whether the TP53 LOH status is unknown, whether the sample is heterozygous for TP53. In one or more examples, the system can determine a feature attribute corresponding to a sample-wide LOH quality control (QC) status category. In one or more examples, the sample-wide LOH quality control (QC) status can include, but is not limited to, whether the sample passes, fails, LOH associated quality controls, or whether the QC status is not applicable.

In one or more examples the system can determine a feature attribute corresponding to a gene co-mutation category for a particular sample. The gene co-mutation category may be indicative of other mutations that are identified in the sample, such that each category corresponds to a different mutation. For example, TP53 variants commonly occur with CDKN2A deletions. In one or more examples, the other mutations may be based on genes that are included in one or more bait sets.

FIG. 2C illustrates exemplary clinical input feature types 210C. As shown in the figure, the clinical level input features can include one or more of an age of the individual, a sex of the individual, an ancestry of the individual, a disease ontology associated with the sample from the individual, and/or the bait set associated with processing of the sample from the individual.

In one or more examples, the clinical features may be determined based on information associated with the medical health records and/or from test requisitions of individuals providing the samples. For example, the system may determine a feature attribute corresponding to an age value of an individual providing a sample of the plurality of samples based on the medical health records of the individual. As another example, the system may determine a feature attribute corresponding to a sex category of the individual based on the medical health records of the individual. For example, the sex category may include, but is not limited to, male, female, and missing. As another example, the system may determine a feature attribute corresponding to an ancestry category of the individual. In one or more examples the ancestry categories can include, but are not limited to, African, Mixed America, East Asian, European, and South Asian, based on the individual's medical health records. As another example, the system may determine a feature attribute corresponding to a disease ontology category of an individual providing a sample of the plurality of samples. In one or more examples the disease ontology may describe one or more of a cancer category (e.g., solid tumor, sarcoma), a region (e.g., thoracic, central nervous system, gastrointestinal), an organ (e.g., lung, brain, stomach). In one or more examples, the disease ontology may relate to a specific disease type (e.g., lung non-small cell lung carcinoma (NSCLC), brain glioma, stomach adenocarcinoma) and/or subtype (e.g., NSCLC not otherwise specified (NOS), brain glioblastoma, stomach adenocarcinoma intestinal type). In one or more examples, the system may determine a feature attribute corresponding to a type of bait set used to process the sample obtained from the individual (e.g., a plasma baitset, a tissue baitset, and the like).

While input feature types and feature attributes have been described with respect to input feature types 210A-210C, a skilled artisan would understand that additional (or fewer) input feature types and additional (or fewer) feature attributes may be used without departing from the scope of this disclosure.

FIG. 2D illustrates feature attribute data corresponding to various input feature types associated with exemplary samples that can be determined at Block 104. For example, at block 104 the system can determine the one or more feature attributes for sample 0 and sample 1. As shown in FIG. 2D, sample 0 may be determined to have a TMB of 2.5, an MSI status of unknown, a minor allele frequency of 0.051, a germline zygosity call, a patient age of 55, and a patient sex of female. Sample 1 may be determined to have a TMB of 25, an MSI status of high, a minor allele frequency of 0.48, a somatic zygosity call, a patient age of 72, and a patient sex of male. While this example illustrates the process of determining feature attributes for samples 0 and 1, A skilled artisan will understand that the feature attributes can be determined for a plurality of samples and for a plurality of input feature types and is not limited to the input feature types shown in FIG. 2D.

At block 106 of FIG. 1, the system can categorize the one or more samples into a plurality of input feature sub-types. In one or more examples, an input feature type may be associated with one or more input feature sub-types. In some instances, the input feature sub-type may correspond to the input feature attributes described above. For example, the sample (e.g., based on the feature attributes associated with the sample) may be categorized based on a categorical sub-type, (e.g., male or female; MSI unknown, MSI high, MSI stable) or a value-based sub-type (e.g., a TMB value, a percent genome LOH value, a LOH ploidy value).

For feature attributes associated with a categorical sub-type, the system may categorize the sample into an appropriate input feature sub-type that corresponds to one of the categorical feature attributes described above. For example, regarding an SV signature input feature, a sample may be determined to correspond to a feature attribute of APOBEC. Accordingly, the sample may be categorized into an APOBEC input feature sub-type associated with the SV signature input feature. As another example, regarding a sex input feature, a sample may be determined to correspond to a feature attribute of male. Such a sample may be categorized into a male input feature sub-type associated with the sex input feature. In one or more examples, the sub-types may be pre-defined based on the input feature types and feature attributes described above. For example, the sex input features may be associated with male, female, and unknown categories, as discussed above. For example, regarding the co-mutations sub-type, if a sample is determined to include one or more mutations (e.g., mutations in TP53, RAF, etc.) the feature attribute would be associated with a corresponding co-mutation category (e.g., a TP53 co-mutation category, a RAF co-mutation category, etc.), as discussed above.

For input features associated with feature attributes corresponding to a value, the system may categorize the sample (e.g., based on a respective feature attribute) into one or more predetermined quantiles based on an expected value for a corresponding input feature. In such embodiments each quantile can correspond to an input feature sub-type. The number of quantiles for each input feature is not intended to limit the scope of this disclosure.

For example, as described above, TMB feature attributes may correspond to a value. In one or more examples, the expected value for a TMB may be in a range of 0-7,000 mutations per megabases detected in a sample. The system may have a number of predetermined quantiles associated with these TMB values, where each quantile corresponds to an input feature sub-type. In one or more examples, the quantiles may be based on regularly spaced intervals (e.g., 0-1,000, 1001-2,000, 2,001-3,000, 3,001-4,000, 4,001-5,000, 5,001-6,000, and 6,001-7,000). In one or more examples the quantiles may be associated with irregularly spaced intervals. (e.g., 0-2.5, 2.6-5.0, 5.1-8.8, 8.9-15.3, 15.4-27.5, 27.6-48.00. 48.01-89.6, and 89.7-7,000.0). In such examples, irregularly spaced intervals may be based in part on an expected distribution of the feature attribute values. That is, the quantiles may be determined such that categorizing the plurality of samples based on their respective feature attribute into the quantiles (e.g., sub-types) provides insight into the distribution of the feature attribute values associated with the input feature.

Comparing the regularly spaced quantiles with the irregularly spaced quantiles provided above, for example, if the feature attribute values for TMB are typically expected to be less than 100, the regularly spaced intervals would not provide insight into the distribution of TMB, at least because most if not all of the values would be categorized into the sub-type of 0-1,000. Meanwhile, the irregularly spaced intervals would likely provide insight into the distribution of TMB, as the sub-types may be designed such that feature attribute values will be distributed amongst the quantiles.

While the quantiles have been described with respect to specific values for TMB, a skilled artisan will understand that the quantiles for TMB are not limited to the example provided above. Additionally, quantiles may be determined for any input feature associated with a numerical value, e.g., an age of an individual, a TMB value, a percent genome LOH, a LOH ploidy, a MAF, and altered copies. In one or more examples, different input features may be associated with a different number of quantiles. For example, the TMB input feature may be associated with a first number of quantiles and the percent genome LOH value may be associated with a second, different number of quantiles.

At block 108 of FIG. 1, the system can obtain one or more input feature values or tallies based on the plurality of input feature sub-types. Accordingly, for a given input feature, each input feature sub-type may correspond to a respective input feature tally. In one or more examples, the input feature tallies may be based on a number or count of samples associated with each sub-type.

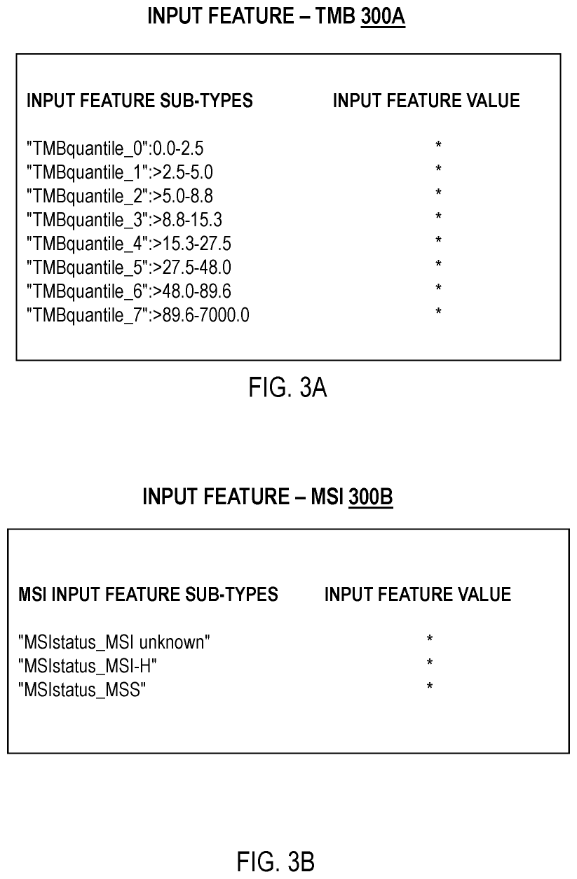

FIG. 3A illustrates exemplary data 300A associated with the TMB input feature. As shown in the figure, there are eight input feature sub-types (e.g., TMB quantile 0, TMB quantile 1, TMB quantile 2, TMB quantile 3, TMB quantile 4, TMB quantile 5, TMB quantile 6, TMB quantile 7) associated with the TMB input feature. Each sub-type is associated with a range of feature attribute values (e.g., a feature attribute determined at block 104). The input feature tally may correspond to a count of samples associated with each input feature sub-type for a given input feature. For example, the system may determine that 500 of the plurality of samples are associated with a TMB value (e.g., feature attribute value) in the range of 0.0-2.5 for the TMB input features. In such examples, the input feature tally associated with TMB quantile 0 would be 500.

FIG. 3B illustrates exemplary data 300B associated with the MSI input feature. As shown in the figure, there are six input feature sub-types (e.g., MSI unknown, MSI high, MSI stable) associated with the MSI input feature. Each sub-type may be associated with a feature attribute determined at block 104. As discussed above, the MSI feature attributes may correspond to one of these pre-determined sub-types. The input feature tally may correspond to a count of samples associated with each input feature sub-type for the plurality of samples. For example, the system may determine that twenty of the plurality of samples are associated with a MSI high status. In such an example, the input feature tally associated with MSI high sub-type would be twenty.

In one or more examples, if the input feature tally for a respective input feature sub-type is below a masking threshold, then the system may re-categorize the samples associated with the input feature sub-type (e.g., corresponding to an input feature tally below the masking threshold) into a masking sub-type. In some examples, the masking sub-type may be specific to a particular input feature.

Creating the masking sub-types may reduce the bias in the data by ensuring that infrequently occurring input feature sub-types are not overrepresented in the data. For example, if for a plurality of samples, there are a low number (e.g., less than the masking threshold) of samples with feature attributes associated with the MSI ambiguous sub-type, the samples associated with the MSI ambiguous sub-type may be assigned to a masking sub-type. In some instances, one or more sub-types may be assigned to the masking sub-type if the respective input feature tally is below the masking threshold. As another example, rare disease ontologies may not be frequently observed in the plurality of samples. Accordingly, such rare disease ontologies may be assigned to a masking sub-type associated with disease ontologies. As another example, infrequently occurring co-mutations may be assigned to a masking sub-type associated with co-mutations. As another example, for input feature sub-types corresponding to quantiles, feature attribute values that fall outside of the predetermined quantiles may be assigned to a masking sub-type.

In some examples, the predetermined threshold to determine whether a feature attribute should be assigned to a masking sub-type may vary by the input feature type, a number of samples being processed (e.g., received at step 102), a number of instances of the sub-type, and the like. In one or more examples, the masking threshold may be large enough not to oversimplify the data (e.g., such that many sub-types are included in the masking sub-type) and prevent the low prevelance of certain features from skewing the statistical model. In one or more examples, the masking threshold may be determined to be around 256.

At block 110 of FIG. 1, the system can input the one or more input feature tallies into a statistical model. In one or more examples, the input feature tallies corresponding to the masking sub-type(s) may also be input into the statistical model. In one or more examples, the statistical model can be a trained machine learning model. For example, the system can input one or more input feature tallies into a trained machine learning model. In one or more examples, the trained machine learning model may be a random forest model.

In one or more examples, the statistical model may be part of a machine learning process. In one or more examples, the machine learning model can include an artificial intelligence (“AI”) learning model. In some instances, the machine learning model can be at least one of a supervised model or an unsupervised model. In one or more examples, the machine learning model can include one or more machine learning models, such as an extreme gradient boosting model, a logistic regression model, an elastic net model, a ridge regression model, a support vector machine model, a naïve-based model, a Gaussian naïve-based model, a limited expectation maximization model, a gradient boosting ensemble model, an adaboost model, a bagging model, a neural network model, a backpropagation model, a stochastic gradient descent model, a non-linear regression model, and a multivariate regression model.

In one or more examples, the model can be trained to predict a functional status of a variant based on an output of the statistical model. In one or more examples, the output of the statistical model may be indicative of a functional status of one or more variants. In one or more examples, the output of the statistical model may include a score indicative of a functional status of a variant as well as values indicative of a relative importance of the input features used to determine the score.

At block 112 of FIG. 1, the system can determine the functional status of a variant based on an output of the statistical model. In one or more examples, the functional status can correspond to a known pathogenic variant, a likely pathogenic variant, a variant of an unknown significance (VUS), and/or a benign variant. In one or more examples, the functional status can correspond to a known pathogenic variant, a variant of an unknown significance (VUS), and a benign variant.

As used herein a known pathogenic variant may correspond to a variant that has a known and documented relevance to disease, e.g., recognized in the scientific literature as being an oncogenic driver of disease. As used herein, a likely pathogenic variant may be relevant to pathogenicity, e.g., as supported by scientific literature, research, and or the genomic data from the one or more samples. As used herein, a VUS may refer to a variant that does not have a well-known or documented association with disease, e.g., there may not be sufficient evidence to identify the variant as a known pathogenic or likely pathogenic variant. As used herein, a benign variant may correspond to a variant that is determined to not be relevant to disease. In some examples, identifying a benign variant may be supported by research and scientific literature.

In one or more examples, the system can predict a functional status of a variant based on one or more outputs of the statistical model. In one or more examples, the score indicative of the functional status may be compared to one or more predefined thresholds to determine the functional status of the variant. In one or more examples, the threshold may be determined during the training process to optimize for certain goals, such as but not limited to, a maximum sensitivity and/or specificity, a maximum negative predictive value with a positive predictive value at a certain minimum etc. In one or more examples, the functional status may be used in reports provided to healthcare providers to provide treatment recommendations.

For example, a healthcare provider may receive a patient sample and run one or more tests on an individual's sample to generate a report. The functional status determination of a variant based on embodiments of this disclosure may be used to provide treatment recommendations. For example, if embodiments of the present disclosure identify a TP53 variant as a known pathogenic alteration, if the patient's sample comprises a TP53 alteration, the report may identify the sample as containing the TP53 alteration and provide treatment recommendations that have been recognized as effective for patients with the TP53 alteration.

In one or more examples, the values indicative of a relative importance of the input features, may be used to provide further information or treatment recommendations in the report. The values indicative of a relative importance of the input features may correspond to a measure of how much poorer the model would have performed the respective features were omitted. For example, the values indicative of a relative importance of the input features may be used to associate a particular variant with certain contexts. As an example, the system may identify the BRCA2 chr13:32910462T>A variant as a likely pathogenic variant based on the output of the model. Accordingly, if a patient's sample is identified as including BRCA2 chr13:32910462T>A, the report may describe that this variant is associated with breast and ovarian cancer. The healthcare provider can determine whether the patient meets the criteria in breast and ovarian cancer to determine the appropriate treatment.

In one or more examples, the values indicative of a relative importance of the input features, may be used to determine which variants a healthcare provider should prioritize when determining treatment options for a patient. For example, a patient's sample may include multiple variants that are identified as known and/or likely pathogenic variants. In such examples, it may be difficult for a healthcare provider to determine which variant(s) should form the basis for treatment selection. For example, if a patient sample includes an EGFR mutation and a BRCA mutation, where both EGFR and BRCA are determined to be known pathogenic variants. In such examples, the values indicative of a relative importance of the input features may be used to determine relationships between these mutations, e.g., whether these mutations typically co-occur, rarely co-occur, whether the EGFR mutation is seen with the BRCA mutation at a higher frequency, and the like.

Based on these relationships, the relative importance values may indicate, for example that the BRCA mutation is typically a passenger gene (e.g., a non-oncogenic driver) when partnered with the EGFR mutation. In such examples, the healthcare provider may base treatment selection on the presence of the EGFR mutation. As another example, the relative importance values of the input features may indicate that EGFR and BRCA typically co-occur and appear to equally drive the disease. In such examples, the healthcare provider may base treatment selection on both the EGFR and BRCA mutations.

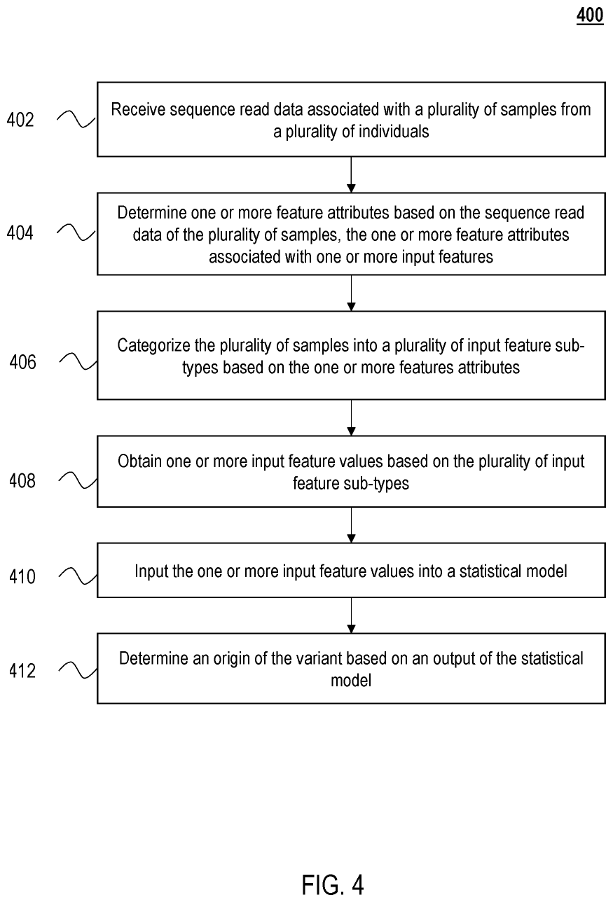

FIG. 4 provides a non-limiting example of an exemplary process 400 for determining an origin of a genomic variant. In one or more examples process 400 may be used to determine an origin of one or more genomic variants. In one or more examples, the origin of a variant may refer to whether a variant is tumor derived or non-tumor derived (e.g., germline or clonal hematopoiesis (CH) derived.

Process 400 can be performed, for example, using one or more electronic devices implementing a software platform. In some examples, process 400 is performed using a client-server system, and the blocks of process 400 are divided up in any manner between the server and a client device. In other examples, the blocks of process 400 are divided up between the server and multiple client devices. Thus, while portions of process 400 are described herein as being performed by particular devices of a client-server system, it will be appreciated that process 400 is not so limited. In other examples, process 400 is performed using only a client device or only multiple client devices. In process 400, some blocks are, optionally, combined, the order of some blocks is, optionally, changed, and some blocks are, optionally, omitted. In some examples, additional steps may be performed in combination with the process 400. Accordingly, the operations as illustrated (and described in greater detail below) are exemplary by nature and, as such, should not be viewed as limiting.

At block 402 of FIG. 4, the system can receive sequence read data associated with a plurality of samples from a plurality of individuals. In some instances, the sequence read data may be associated with one or more genomic variants in the samples. In one or more examples, the block 402 may correspond to the description provided above with respect to block 102.

At block 404 of FIG. 4, the system can determine one or more feature attributes associated with one or more input features based on the sequence read data of the plurality of samples. In one or more examples, the input features may include variant level features (e.g., features characterizing a particular variant identified in a sample), sample level features (e.g., features characterizing a particular sample), and clinical features (e.g., features associated with the individual, disease, and/or test). In one or more examples, additional information obtained via an individual's medical health records and/or test requisition may be used to determine the one or more feature attributes. In one or more examples, the feature attributes may correspond to a value associated with an input feature and/or one or more categories associated with the input features. In one or more examples, block 404 may correspond to the description provided above with respect to block 104. Exemplary input feature types may correspond to the description provided above with respect to FIGS. 2A-2C.

At block 406 of FIG. 4, the system can categorize the plurality of samples into a plurality of input feature sub-types, as described above with respect to block 106 of FIG. 1. In one or more examples, an input feature may be associated with one or more input feature sub-types. For example, an input feature may be associated with a categorical sub-type, where the system categorizes a sample (e.g., based on a categorical feature attribute) into an appropriate input feature sub-type (e.g., the sex input feature may be associated with male and female sub-types; the MSI input feature may be associated with a MSI unknown, a MSI high, a MSI stable). As another example, an input feature may be associated with a value-based subtype, where the system categorizes the sample into one or more quantiles based on a value of the feature attribute determined at step 104 (e.g., a TMB value, a percent genome LOH value, a LOH ploidy value, etc.).

At block 408 of FIG. 4, the system can obtain one or more input feature tallies or values based on the plurality of input feature sub-types, as described above with respect to block 108 of FIG. 1. Each input feature sub-type may correspond to a respective input feature tally. In one or more examples, the input feature tallies may correspond to a count (e.g., a number) of samples associated with each sub-type, as described above with respect to FIGS. 3A-3B.

At block 410 of FIG. 4, the system can input the one or more input feature tallies into a statistical model, as described above with respect to block 110 of FIG. 1. In one or more examples, the statistical model can be a trained machine learning model. For example, the system can input one or more of the one or more input feature tallies into a trained machine learning model. In one or more examples, the trained machine learning model may be a random forest model. In one or more examples, the model can be trained to predict an origin of a variant based on an output of the statistical model. In one or more examples, the output of the statistical model may be indicative of origins of one or more variants. In one or more examples, the output of the statistical model may include a score indicative of an origin of a variant as well as values indicative of a relative importance of the input features used to determine the score.

At block 412 of FIG. 4, the system can determine the origin of a variant based on an output of the statistical model. In one or more examples, the origin can correspond to whether the variant is tumor derived or non-tumor derived (e.g., germline or CH-derived). In one or more examples, the system can predict an origin of a variant based on one or more outputs of the statistical model.

In one or more examples, an output of the statistical model may correspond to a score indicative of an origin of the variant. The score indicative of the origin of the variant may be compared to one or more predefined thresholds to determine the origin of the variant. In one or more examples, the origin may be used in reports provided to healthcare providers to provide treatment recommendations.

For example, a healthcare provider may receive a patient sample and run one or more tests on an individual's sample to generate a report. The origin determination of a variant based on embodiments of this disclosure may be used to provide treatment recommendations. For example, if embodiments of the present disclosure identify two or more variants in a patient's sample, the healthcare provider may select which variant a treatment regimen should be based upon. If one of these variants is identified as non-tumor derived (e.g., CH-derived), the report may identify such variants as being non-tumor derived. Accordingly, the healthcare provider may determine that the treatment regimen should be based on the other variant(s).

In one or more examples, the values indicative of a relative importance of the input features, may be used to provide further information or treatment recommendations in the report. For example, the values indicative of a relative importance of the input features may be used to associate a particular variant with certain contexts. As an example, according to process 400, a variant for a particular gene may be determined to be more likely to be tumor-derived in younger patients. Thus, if a sample is determined to include this variant and that the patient is younger, the system and/or healthcare provider may determine that the variant is likely to be tumor-derived in this context.

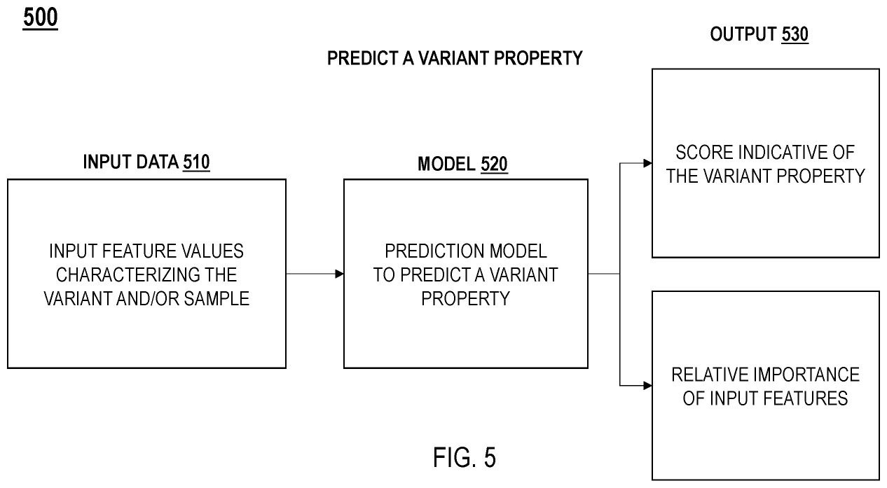

FIG. 5 is a diagram illustrating a process of predicting a variant property using a statistical model, according to embodiments of the present disclosure. As shown in the figure, input data 510 corresponding to one or more input feature tallies associated with one or more input features (e.g., variant input feature types 210A, sample input feature types 210B, and clinical input feature types 210C) can be input into model 520. In one or more examples, the input data 510 can be associated with blocks 104-108 in FIG. 1, and FIGS. 2A-2D, 3A, and 3B described above.

The model 520 can be a statistical model, such as a trained machine learning model configured to predict determine a variant property (e.g., functional status or origin) based on a plurality of samples as described above with respect to process 100 and 400. The model 520 can then output 530 one or more scores indicative of a receptor status. As shown in the figure, the output 530 of the model can include one or more scores indicative of the variant property. For example, if the variant property corresponds to the functional status, the score can be indicative of the pathogenicity of the variant. If the variant property corresponds to an origin of the variant, the score can be indicative of whether the variant is tumor-derived. In one or more examples, the model 520 may further output a relative importance of the input features. The relative importance of the input features may be expressed as a percentage contribution of each of the input feature types and/or input feature sub-types to the score. The relative importance of the input features may be expressed in other ways (e.g., ranking feature types and/or input feature sub-types from most important to least important) without departing from the scope of this disclosure.

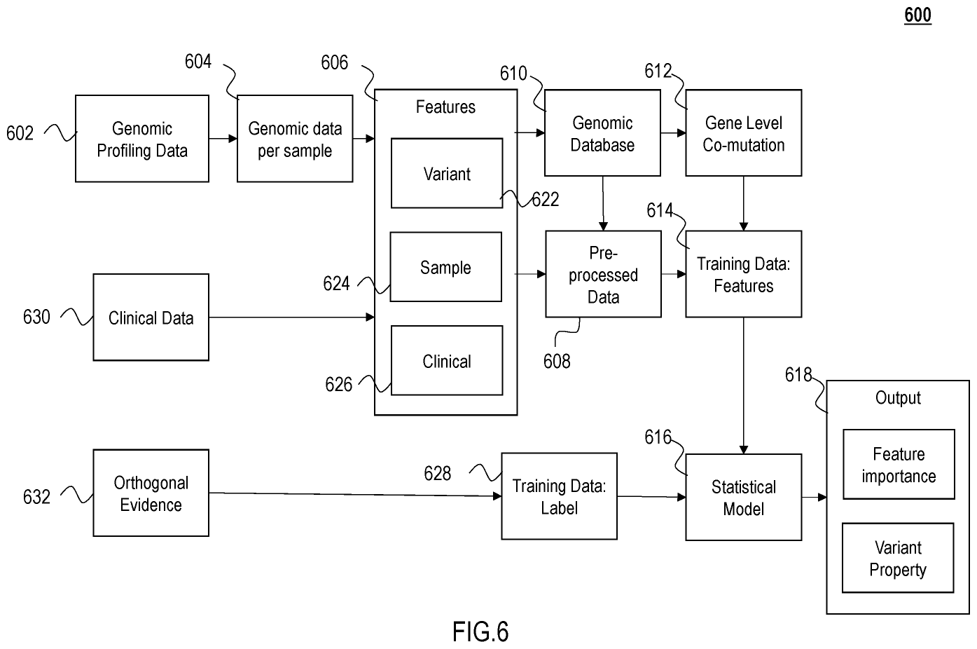

FIG. 6 provides a non-limiting example of an exemplary process 600 for producing a statistical model for identifying a variant property, e.g., a functional status or an origin of the variant. As shown in the figure, the process 600 may use supervised learning to create the statistical model.

Process 600 can be performed, for example, using one or more electronic devices implementing a software platform. In some examples, process 600 is performed using a client-server system, and the blocks of process 600 are divided up in any manner between the server and a client device. In other examples, the blocks of process 600 are divided up between the server and multiple client devices. Thus, while portions of process 600 are described herein as being performed by particular devices of a client-server system, it will be appreciated that process 600 is not so limited. In other examples, process 600 is performed using only a client device or only multiple client devices. In process 600, some blocks are, optionally, combined, the order of some blocks is, optionally, changed, and some blocks are, optionally, omitted. In some examples, additional steps may be performed in combination with the process 600. Accordingly, the operations as illustrated (and described in greater detail below) are exemplary by nature and, as such, should not be viewed as limiting.

At block 602, the system can receive genomic profiling data corresponding to a plurality of samples. In one or more examples, the genomic profiling data can correspond to comprehensive genomic profiling (CGP). In one or more examples, the genomic profiling data can correspond to targeted sequencing. In one or more examples, the genomic profiling data received at block 602 may be associated with the sequence read data described with respect to block 102 of FIG. 1. At block 630, the system can receive clinical data associated with the genomic profiling data, e.g., from one or more test requisitions or medical health records associated with individuals providing the samples.

At block 604, the system may analyze the genomic data on a per sample basis. For example, the system may analyze the genomic data for one or more samples of the plurality of samples received at block 602. In one or more examples, the system may analyze the genomic data for each sample of the plurality of samples.

At block 606, the system can determine various feature attributes for a plurality of features based on the genomic profiling data and the clinical data for each sample. The feature attributes may correspond to specific values or categories that characterize a respective feature. For example, a feature attribute of “male” may be associated with and characterize a “sex” input feature type.

In one or more examples, the features 606 may include variant features 622 (e.g., features characterizing a particular variant identified in a sample), sample features 624 (e.g., features characterizing a particular sample), and clinical features 626 (e.g., features associated with the individual, disease, and/or test). In one or more examples, the system can determine feature attributes related to one or more variant features 622, sample features 624, and clinical features 626. In one or more examples, the feature attributes determined with respect to features 606 may be determined in a similar manner as described above with respect to block 104. For example, the features 606 may correspond to the input feature types 210A-210C and respective feature attributes described above.

At block 608, the system can pre-process the features (e.g., variant features 622, sample features 624, and clinical features 626) to generate training features at block 614. In one or more examples, pre-processing the features can include categorizing the samples (e.g., based on their respective feature attributes) into a plurality of input feature sub-types, as described above with respect to blocks 106 and 108.

For example, an input feature type, such as a sex of an individual, may be associated with one or more input feature sub-types, such as “male” or “female.” As described above, the input feature attributes may be associated with a category, (e.g., male or female; MSI unknown, MSI high, MSI stable, etc.) or a value (e.g., a TMB value, a percent genome LOH value, a LOH ploidy value). For feature attributes associated with a category, the system may categorize a sample (e.g., based on its respective feature attribute) into an appropriate feature sub-type. For feature attributes associated with a value, the system can categorize the sample into one or more predetermined quantiles based on the value of the feature attribute. In such embodiments each quantile corresponds to an input feature sub-type.

At block 610, the system may aggregate the features and use a genomic database to determine one or more gene co-mutations for a variant in a sample at block 612. The gene co-mutations may correspond to a count of co-mutating pathogenic variants based on one or more baited genes from the genetic profiling data. The gene co-mutations may be organized by variant type, such that each variant is associated with a separate co-mutation value. In one or more examples, the gene co-mutations determined at block 612 can be used to generate one or more training features 614. For example, the co-mutation value corresponding to a count of a particular co-mutating variant may correspond to a training input feature. Accordingly, the training features produced at block 614 may be based on the pre-processed input features and the gene level co-mutation. In one or more examples, information from the genomic database may be used to pre-process the features at block 608, as described above.

At block 616, the training features may be input into the statistical model for training the statistical model. In one or more examples, the statistical model may be trained to identify a variant property (e.g., functional status or origin) associated with a particular variant. For example, the statistical model may be configured to determine the functional status for a TP53 variant. In one or more examples, the statistical model may be trained with an 80:20 training-validation split and supervised random forest learning to identify a property (e.g., functional status or origin) associated with one or more variants.

In one or more examples, the machine learning model may be a random forest model. In one or more examples, the machine learning model can include one or more machine learning models, such as an extreme gradient boosting model, a logistic regression model, an elastic net model, a ridge regression model, a support vector machine model, a naïve-based model, a Gaussian naïve-based model, a limited expectation maximization model, a gradient boosting ensemble model, an adaboost model, a bagging model, a neural network model, a backpropagation model, a stochastic gradient descent model, a non-linear regression model, and a multivariate regression model.

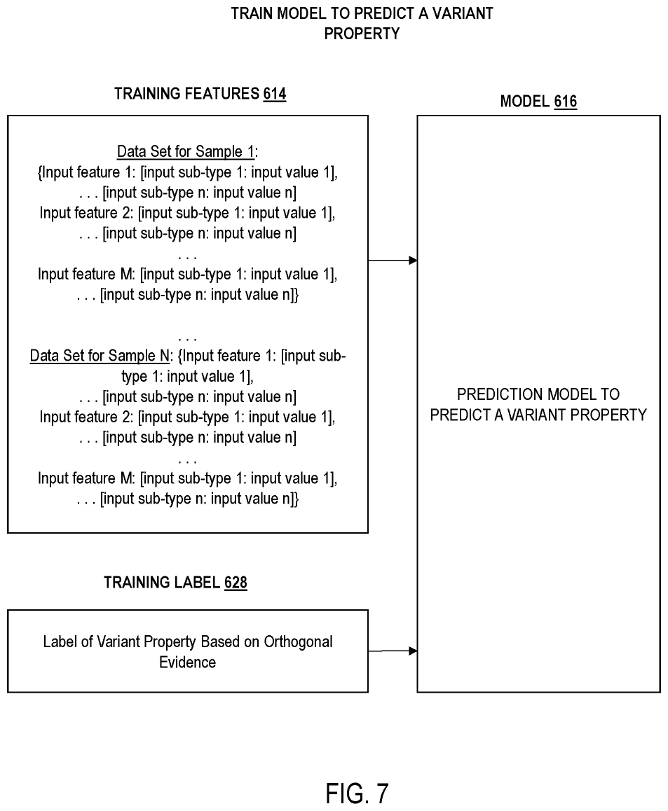

In one or more examples, the statistical model is trained via supervised learning. Accordingly, in one or more examples, the training data determined at block 614 can be associated with a respective label. FIG. 7 provides a diagram illustrating exemplary training features 614 and an exemplary training label 628 being input into a model 616 to predict a variant property. As shown in the figure, the training features 614 can include n input values or tallies associated with input sub-types for a plurality of M input features. The training label 628 may be based on orthogonal evidence.

Referring back to FIG. 6, at block 628 the label can be determined by orthogonal evidence received by the system (at block 632). The label may be indicative of a functional status of the variant associated with the statistical model. For example, if the statistical model is configured to determine a functional status for a TP53 variant, the label may correspond to the predicted functional status of TP53 based on one or more databases and scientific literature. For example, the system may receive an indication of a functional status of the variant based on data from one or more databases (e.g., ONCOKB™, cBioPortal, CIViC, ClinVar, COSMIC, and gnomAD) and/or scientific literature that the TP53 variant is a known pathogenic variant. The label may be used as a ground truth to train the statistical model.

As another example, the statistical model may be configured to determine an origin of a TP53 variant. In such examples, the system may determine the label based on orthogonal evidence including, but not limited to one or more databases (e.g., ONCOKB™, cBioPortal, CIViC, ClinVar, COSMIC, and gnomAD) and/or scientific literature. The label may be used as a ground truth to train the statistical model.

At block 618, the statistical model can output at least a variant property (e.g., functional status or origin) and/or indicate the relative feature importance of the features used to determine the variant property.

In one or more examples, variant property can correspond to a functional status where the functional status can correspond to a known pathogenic variant, a likely pathogenic variant, a variant of an unknown significance (VUS), and a benign variant, as described above with respect to block 112. In one or more examples, the functional status can correspond to a known pathogenic variant, a variant of an unknown significance (VUS), and a benign variant. In such examples, the relative feature importance may correspond to a ranking of one or more features used to determine the functional status, as described above with respect to block 112.

In one or more examples, the variant property can correspond to an origin of the variant, where the origin can correspond to a tumor-derived variant, or non-tumor derived variant (e.g., germline-derived variant or a clonal hematopoiesis (CH) derived variant) as described above with respect to block 412.

In one or more examples, the relative importance of the input features may be expressed as a percentage contribution of each of the input features and/or input feature sub-types to the score. The relative importance of the input features may be expressed in other ways (e.g., ranking features from most important to least important) without departing from the scope of this disclosure. In one or more examples, the relative feature importance of the features may be used to select features to include in the trained statistical model, thereby refining the model.

In some instances, the disclosed methods may be used to identify variant properties for variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify variant properties for variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining a variant property based on a plurality of samples may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining a variant property based on a plurality of samples may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining a variant property based on a plurality of samples may be used to select a subject (e.g., a patient) for a clinical trial based on the output indicative of the variant property determined for one or more gene loci. In some instances, patient selection for clinical trials based on, e.g., identification of a variant property based on one or more gene loci, may accelerate the development of targeted therapies and improve the healthcare outcomes for treatment decisions.

In some instances, the disclosed methods for determining a variant property based on a plurality of samples may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for determining a variant property based on a plurality of samples may be used in treating a disease (e.g., a cancer) in a subject. For example, in response to determining the variant property based on a plurality of samples using any of the methods disclosed herein, an effective amount of an anti-cancer therapy or anti-cancer treatment may be administered to the subject.

In some instances, the disclosed methods for determining a variant property based on a plurality of samples may be used for monitoring disease progression or recurrence (e.g., cancer or tumor progression or recurrence) in a subject. For example, in some instances, the methods may be used to determine the variant property based on a plurality of samples in a first sample obtained from the subject at a first time point, and used to determine the variant property based on a plurality of samples in a second sample obtained from the subject at a second time point, where comparison of the first determination of the variant property and the second determination of the variant property allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the variant property based on a plurality of samples.

In some instances, the disclosed methods for determining a variant property based on a plurality of samples may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a variant property based on a plurality of samples as part of a genomic profiling process (or inclusion of the output from the disclosed methods for determining a variant property based on a plurality of samples as part of the genomic profile of the subject) can improve the validity of, e.g., disease detection calls and treatment decisions, made on the basis of the genomic profile by, for example, independently confirming a variant property (e.g., a functional status or variant origin) in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or nonspecific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (See, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for determining a variant property based on a plurality of samples (e.g., a functional status of a variant or an origin of a variant). The systems may comprise, e.g., one or more processors, and a memory unit communicatively coupled to the one or more processors and configured to store instructions that, when executed by the one or more processors, cause the system to: receive, using one or more processors, sequence read data associated with a plurality of samples; determining, using the one or more processors, one or more feature attributes associated with one or more input features based on the sequence read data of the plurality of samples; categorizing, using the one or more processors, the one or more samples into a plurality of input feature sub-types, wherein an input feature of the one or more input features is associated with one or more input feature sub-types of the plurality of input feature sub-types; obtaining, using the one or more processors, one or more input feature tallies based on the plurality of input feature sub-types; inputting, using the one or more processors, the one or more input feature tallies into a statistical model; and determining, using the one or more processors, the variant property of the variant based on an output of the statistical model, wherein the variant property is indicative of a functional status of the variant or an origin of the variant. In one or more examples, the functional status can be indicative of a level of pathogenicity of the variant. In one or more examples, the origin of the variant can be indicative of whether the variant is tumor-derived or non-tumor derived (e.g., germline or CH-derived).

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining a variant property based on a variety of samples as described herein (e.g., a tissue sample, biopsy sample, hematological sample, or liquid biopsy sample derived from the subject).

In some instances, the plurality of gene loci for which sequencing data is processed to determine the variant property may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of the variant property is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

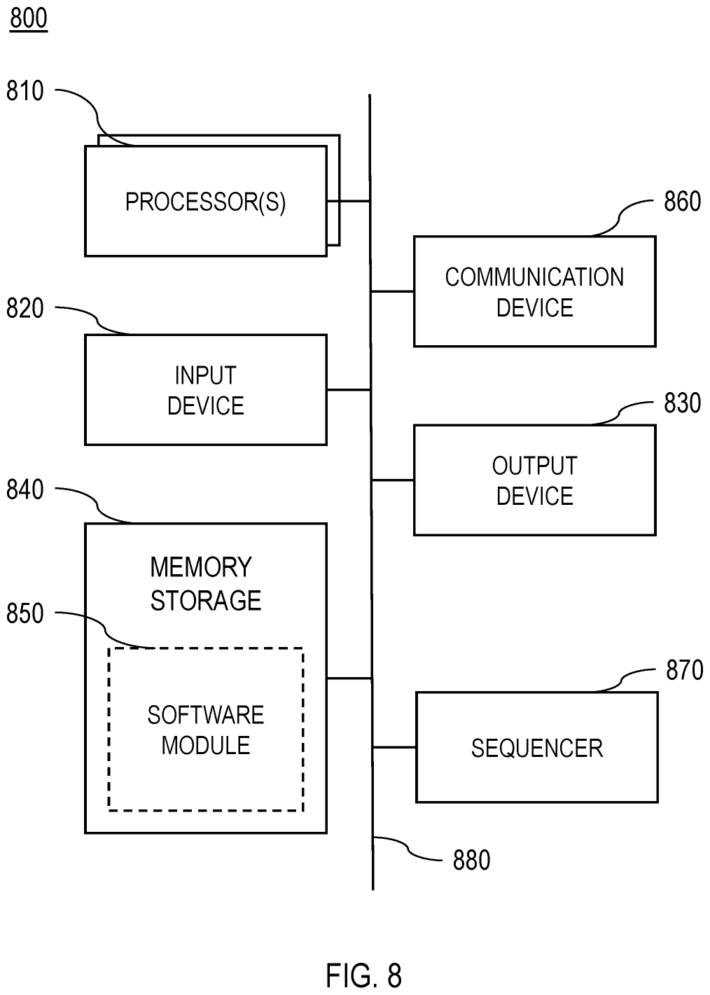

FIG. 8 illustrates an example of a computing device or system in accordance with one embodiment. Device 800 can be a host computer connected to a network. Device 800 can be a client computer or a server. As shown in FIG. 8, device 800 can be any suitable type of microprocessor-based device, such as a personal computer, workstation, server or handheld computing device (portable electronic device) such as a phone or tablet. The device can include, for example, one or more processor(s) 810, input devices 820, output devices 830, memory or storage devices 840, communication devices 860, and nucleic acid sequencers 870. Software 850 residing in memory or storage device 840 may comprise, e.g., an operating system as well as software for executing the methods described herein. Input device 820 and output device 830 can generally correspond to those described herein, and can either be connectable or integrated with the computer.

Input device 820 can be any suitable device that provides input, such as a touch screen, keyboard or keypad, mouse, or voice-recognition device. Output device 830 can be any suitable device that provides output, such as a touch screen, haptics device, or speaker.

Storage 840 can be any suitable device that provides storage (e.g., an electrical, magnetic or optical memory including a RAM (volatile and non-volatile), cache, hard drive, or removable storage disk). Communication device 860 can include any suitable device capable of transmitting and receiving signals over a network, such as a network interface chip or device. The components of the computer can be connected in any suitable manner, such as via a wired media (e.g., a physical system bus 880, Ethernet connection, or any other wire transfer technology) or wirelessly (e.g., Bluetooth®, Wi-Fi®, or any other wireless technology).

Software module 850, which can be stored as executable instructions in storage 840 and executed by processor(s) 810, can include, for example, an operating system and/or the processes that embody the functionality of the methods of the present disclosure (e.g., as embodied in the devices as described herein).

Software module 850 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 840, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

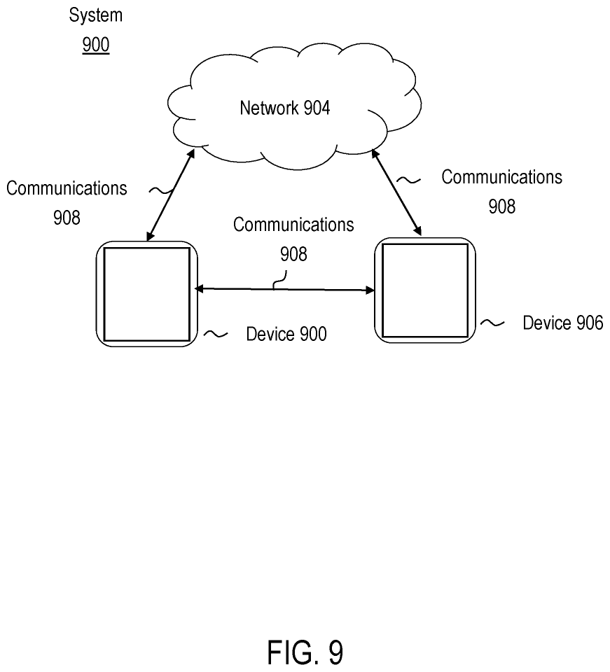

Device 800 may be connected to a network (e.g., network 904, as shown in FIG. 9 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 800 can be implemented using any operating system, e.g., an operating system suitable for operating on the network. Software module 850 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 810.

Device 800 can further include a sequencer 870, which can be any suitable nucleic acid sequencing instrument.

FIG. 9 illustrates an example of a computing system in accordance with one embodiment. In system 900, device 800 (e.g., as described above and illustrated in FIG. 8) is connected to network 904, which is also connected to device 906. In some embodiments, device 906 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 800 and 906 may communicate, e.g., using suitable communication interfaces via network 904, such as a Local Area Network (LAN), Virtual Private Network (VPN), or the Internet. In some embodiments, network 904 can be, for example, the Internet, an intranet, a virtual private network, a cloud network, a wired network, or a wireless network. Devices 800 and 906 may communicate, in part or in whole, via wireless or hardwired communications, such as Ethernet, IEEE 802.11b wireless, or the like. Additionally, devices 800 and 906 may communicate, e.g., using suitable communication interfaces, via a second network, such as a mobile/cellular network. Communication between devices 800 and 906 may further include or communicate with various servers such as a mail server, mobile server, media server, telephone server, and the like. In some embodiments, Devices 800 and 906 can communicate directly (instead of, or in addition to, communicating via network 904), e.g., via wireless or hardwired communications, such as Ethernet, IEEE 802.11b wireless, or the like. In some embodiments, devices 800 and 906 communicate via communications 908, which can be a direct connection or can occur via a network (e.g., network 904).

One or all of devices 800 and 906 generally include logic (e.g., http web server logic) or are programmed to format data, accessed from local or remote databases or other sources of data and content, for providing and/or receiving information via network 904 according to various examples described herein.

### EXEMPLARY IMPLEMENTATIONS

Exemplary implementations of the methods and systems described herein include:
1. A method comprising:
   providing a plurality of nucleic acid molecules obtained from a plurality of samples from a plurality of subjects;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads associated with the plurality of samples;
   determining, using the one or more processors, one or more feature attributes based on the sequence read data of the plurality of samples, the one or more feature attributes associated with one or more input features;
   categorizing, using the one or more processors, the plurality of samples into a plurality of input feature sub-types based on the one or more feature attributes, wherein an input feature of the one or more input features is associated with one or more input feature sub-types of the plurality of input feature sub-types;
   obtaining, using the one or more processors, one or more input feature values based on the plurality of input feature sub-types;
   inputting, using the one or more processors, the one or more input feature values into a statistical model; and
   determining, using the one or more processors, the functional status of a variant based on an output of the statistical model.
2. The method of clause 1, wherein the functional status is indicative of a level of pathogenicity of the variant.
3. The method of clause 1 or clause 2, wherein the non-known pathogenic status comprises a likely pathogenic status of the variant, a variant of unknown significance status of the variant, a benign status of the variant, or a combination thereof.
4. The method of any one of clauses 1 to 3, wherein the plurality of input feature sub-types correspond to pre-determined sub-types.
5. The method of any of clauses 1 to 4, further comprising organizing a feature attribute of the one or more feature attributes into a first category based on a first input feature if a number of samples associated with the first input feature is below a pre-determined threshold.
6. The method of any of clauses 1 to 5, wherein the one or more input features are associated with one or more variant features, one or more sample features, one or more clinical features, or a combination thereof.
7. The method of any of clauses 1 to 6, wherein the output of the statistical model is indicative of a functional status of the variant.
8. The method of any of clauses 1 to 7, wherein the output of the statistical model comprises one or more feature scores indicative of a relative importance of the one or more input features.
9. The method of any of clauses 1 to 8, wherein the statistical model is a trained machine learning model and trained by:
   receiving, using the one or more processors, training data including one or more training feature values based on one or more training feature sub-types associated with a plurality of training samples; and
   training, using the one or more processors, the machine learning model based on the training data.
10. The method of clause 9, wherein the one or more training feature sub-types are associated with one or more training features, the one or more training features comprising: one or more variant features, one or more sample features, one or more clinical features, or a combination thereof.
11. The method of any of clause 9 or clause 10, further comprising obtaining training data, comprising:
   receiving, using one or more processors, training sequence read data associated with the plurality of training samples;
   determining, using the one or more processors, one or more training feature attributes based on the training sequence read data;
   organizing, using the one or more processors, the one or more training feature attributes into the one or more training feature sub-types;
   obtaining the one or more training feature values based on the one or more training feature sub-types;
   inputting, using the one or more processors, the one or more training feature values into an untrained statistical model;
   predicting, using the one or more processors, the functional status of the variant based on the one or more training feature values;
   obtaining one or more training feature scores indicative of a relative importance of the one or more training features;
   updating one or more weights associated with a trained statistical model based on the one or more training feature scores.
12. The method of clause 11, further comprising organizing a training feature attribute of the one or more training feature attributes into a first category based on a first training feature, if a number of training samples of the plurality of training samples associated with the first training feature is below a pre-determined threshold.
13. The method of any one of clauses 1 to 12, wherein the subject is suspected of having or is determined to have cancer.
14. The method of clause 13, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
15. The method of clause 13, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
16. The method of clause 15, further comprising treating the subject with an anti-cancer therapy.
17. The method of clause 16, wherein the anti-cancer therapy comprises a targeted anti-cancer therapy.
18. The method of clause 17, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
19. The method of any one of clauses 1 to 18, further comprising obtaining the plurality of samples from the plurality of subjects.
20. The method of any one of clauses 1 to 19, wherein a sample of the plurality of samples comprises a tissue biopsy sample, a liquid biopsy sample, or a normal control.
21. The method of clause 20, wherein the sample is a liquid biopsy sample and comprises blood, plasma, cerebrospinal fluid, sputum, stool, urine, or saliva.
22. The method of clause 20, wherein the sample is a liquid biopsy sample and comprises circulating tumor cells (CTCs).
23. The method of clause 20, wherein the sample is a liquid biopsy sample and comprises cell-free DNA (cfDNA), circulating tumor DNA (ctDNA), or any combination thereof.
24. The method of any one of clauses 1 to 23, wherein the plurality of nucleic acid molecules comprises a mixture of tumor nucleic acid molecules and non-tumor nucleic acid molecules.
25. The method of clause 24, wherein the tumor nucleic acid molecules are derived from a tumor portion of a heterogeneous tissue biopsy sample, and the non-tumor nucleic acid molecules are derived from a normal portion of the heterogeneous tissue biopsy sample.
26. The method of clause 24, wherein a sample of the plurality of samples comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
27. The method of any one of clauses 1 to 26, wherein the one or more adapters comprise amplification primers, flow cell adaptor sequences, substrate adapter sequences, or sample index sequences.
28. The method of any one of clauses 1 to 27, wherein the captured nucleic acid molecules are captured from the amplified nucleic acid molecules by hybridization to one or more bait molecules.
29. The method of clause 28, wherein the one or more bait molecules comprise one or more nucleic acid molecules, each comprising a region that is complementary to a region of a captured nucleic acid molecule.
30. The method of any one of clauses 1 to 29, wherein amplifying nucleic acid molecules comprises performing a polymerase chain reaction (PCR) amplification technique, a non-PCR amplification technique, or an isothermal amplification technique.
31. The method of any one of clauses 1 to 30, wherein the sequencing comprises use of a massively parallel sequencing (MPS) technique, whole genome sequencing (WGS), whole exome sequencing, targeted sequencing, direct sequencing, or Sanger sequencing technique.
32. The method of clause 31, wherein the sequencing comprises massively parallel sequencing, and the massively parallel sequencing technique comprises next generation sequencing (NGS).
33. The method of any one of clauses 1 to 32, wherein the sequencer comprises a next generation sequencer.
34. The method of any one of clauses 1 to 33, wherein one or more of the plurality of sequencing reads overlap one or more gene loci within one or more subgenomic intervals in a sample of the plurality of samples.
35. The method of clause 34, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
36. The method of clause 34 or clause 35, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
37. The method of clause 34 or clause 35, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.
38. The method of any one of clauses 1 to 37, further comprising generating, by the one or more processors, a report indicating the functional status of the variant.
39. The method of clause 38, further comprising transmitting the report to a healthcare provider.
40. The method of clause 39, wherein the report is transmitted via a computer network or a peer-to-peer connection.
41. A method for determining a functional status of a variant, the method comprising:
   receiving, using one or more processors, sequence read data associated with a plurality of samples;
   determining, using the one or more processors, one or more feature attributes based on the sequence read data of the plurality of samples, the one or more feature attributes associated with one or more input features;
   categorizing, using the one or more processors, the plurality of samples into a plurality of input feature sub-types based on the one or more feature attributes, wherein an input feature of the one or more input features is associated with one or more input feature sub-types of the plurality of input feature sub-types;
   obtaining, using the one or more processors, one or more input feature values based on the plurality of input feature sub-types;
   inputting, using the one or more processors, the one or more input feature values into a statistical model; and
   determining, using the one or more processors, the functional status of the variant based on an output of the statistical model,
   wherein the functional status is indicative of a level of pathogenicity of the variant.
42. The method of any of clause 41, wherein the functional status comprises a known pathogenic status of the variant or a non-known pathogenic status of the variant.
43. The method of clause 42, wherein the non-known pathogenic status comprises a likely pathogenic status of the variant, a variant of unknown significance status of the variant, a benign status of the variant, or a combination thereof.
44. The method of clause 42, wherein the non-known pathogenic status comprises a variant of unknown significance status of the variant, a benign status of the variant, or a combination thereof.
45. The method of any of clauses 41 to 44, wherein the plurality of input feature sub-types correspond to pre-determined sub-types.
46. The method of clause 45, wherein one or more of the pre-determined sub-types comprise quantiles based on the one or more input features.
47. The method of any of clauses 41 to 46, further comprising:
   inputting, using the one or more processors, the one or more feature attributes into a genomic database;
   determining, using the one or more processors, a gene co-mutation value indicative of a prevalence of gene co-mutations associated with the variant based on the genomic database; and
   inputting, using the one or more processors, the gene co-mutation value into the statistical model,
   wherein determining the functional status is based on the gene co-mutation value.
48. The method of any of clauses 41 to 47, further comprising organizing a feature attribute of the one or more feature attributes into a first category based on a first input feature if a number of samples associated with the first input feature is below a pre-determined threshold.
49. The method of any of clauses 41 to 48, wherein the one or more input features are associated with one or more variant features, one or more sample features, one or more clinical features, or a combination thereof.
50. The method of clause 49, further comprising obtaining clinical information, wherein determining the one or more input features is further based on the clinical information.
51. The method of any of clause 49 or clause 50, wherein the one or more variant features comprise a presence of a short variant, an absence of a short variant, a variant minor allele frequency, a germline status, a somatic status, a zygosity determination, a copy number alteration, a genomic rearrangement, or a combination thereof.
52. The method of any of clauses 49 to 51, wherein the one or more sample features comprise a bait-set, a tumor purity, a loss of heterozygosity (LOH) status, a LOH ploidy status, a LOH TP53 status, a LOH QC status, a microsatellite instability, a tumor mutational burden, a mutational signature, a gene co-mutation, or a combination thereof.
53. The method of any of clauses 49 to 52, wherein the one or more clinical features comprise an age of the individual, a sex of the individual, a disease ontology, a genomic ancestry of the individual, a bait set, or a combination thereof.
54. The method of any of clauses 41 to 53, wherein the output of the statistical model is indicative of a functional status of the variant.
55. The method of any of clauses 41 to 54, wherein the output of the statistical model comprises one or more feature scores indicative of a relative importance of the one or more input features.
56. The method of any of clauses 41 to 55, wherein the statistical model is a trained machine learning model and trained by:
   receiving, using the one or more processors, training data including one or more training feature values based on one or more training feature sub-types associated with a plurality of training samples; and
   training, using the one or more processors, the machine learning model based on the training data.
57. The method of clause 56, wherein the one or more training feature sub-types are associated with one or more training features, the one or more training features comprising: one or more variant features, one or more sample features, one or more clinical features, or a combination thereof.
58. The method of any of clauses 56 to 57, further comprising obtaining training data, comprising:
   receiving, using one or more processors, training sequence read data associated with the plurality of training samples;
   determining, using the one or more processors, one or more training feature attributes based on the training sequence read data;
   organizing, using the one or more processors, the one or more training feature attributes into the one or more training feature sub-types;
   obtaining the one or more training feature values based on the one or more training feature sub-types;
   inputting, using the one or more processors, the one or more training feature values into an untrained statistical model;
   predicting, using the one or more processors, the functional status of the variant based on the one or more training feature values;
   obtaining one or more training feature scores indicative of a relative importance of the one or more training features;
   updating one or more weights associated with a trained statistical model based on the one or more training feature scores.
59. The method of clause 58, further comprising organizing a training feature attribute of the one or more training feature attributes into a first category based on a first training feature, if a number of training samples of the plurality of training samples associated with the first training feature is below a pre-determined threshold.
60. The method of any of clauses 58 to 59, further comprising:
   inputting, using the one or more processors, the one or more training feature attributes into a genomic database;
   determining, using the one or more processors, a gene co-mutation value indicative of a number of gene co-mutations associated with the variant based on the genomic database; and
   inputting, using the one or more processors, the gene co-mutation value into the untrained statistical model,
   wherein predicting the functional status is further based on the gene co-mutation value.
61. The method of any of clauses 58 to 60, further comprising:
   obtaining a pre-defined functional status of the variant based on an orthogonal method;
   labeling the variant based on the pre-defined functional status; and
   inputting the labeled pre-defined functional status of the variant into the untrained statistical model,
   wherein updating one or more weights is based on the labeled pre-defined functional status.
62. The method of clause 61, wherein the orthogonal method is based on variants identified in literature, variants identified in catalogue of somatic mutations in cancer (COSMIC), or a combination thereof.
63. The method of any of clauses 41 to 62, wherein the statistical model is a machine learning model.
64. The method of any of clauses 41 to 63, wherein the statistical model is part of a machine learning process.
65. The method of any of clauses 41 to 64, wherein the statistical model includes an artificial intelligence learning model.
66. The method of any of clauses 41 to 65, wherein the statistical model comprises a random forest model.
67. The method of any of clauses 41 to 66, wherein the statistical model comprises at least one of an extreme gradient boosting model, a logistic regression model, an elastic net model, a ridge regression model, a support vector machine model, a naïve-based model, a Gaussian naïve-based model, a limited expectation maximization model, a gradient boosting ensemble model, an adaboost model, a bagging model, a neural network model, a backpropagation model, a stochastic gradient descent model, a non-linear regression model, and a multivariate regression model.
68. The method any of clauses 41 to 67, further comprising reclassifying, using the one or more processors, the variant based on the functional status.
69. The method any of clauses 41 to 68, further comprising generating, using the one or more processors, a report comprising the functional status of the variant.
70. The method any of clauses 41 to 69, further comprising assigning, using the one or more processors, a therapy for an individual based on the functional status.
71. The method any of clauses 41 to 70, further comprising determining, using the one or more processors, a treatment decision for an individual based on the functional status.
72. The method any of clauses 41 to 71, wherein the method further comprises selecting an anti-cancer therapy to administer to the subject based on the determination of the functional status.
73. The method any of clauses 41 to 72, further comprising administering, using the one or more processors, a treatment to an individual based on the functional status.
74. The method any of clauses 41 to 73, wherein the method further comprises determining an effective amount of an anti-cancer therapy to administer to the subject based on the determination of the functional status.
75. The method of clause 74, wherein the anti-cancer therapy comprises chemotherapy, radiation therapy, immunotherapy, a targeted therapy, or surgery.
76. The method any of clauses 41 to 75, further comprising monitoring, using the one or more processors, a prognosis of an individual based on the functional status.
77. The method any of clauses 41 to 76, further comprising predicting, using the one or more processors, one or more clinical outcomes based on the functional status.
78. The method any of clauses 41 to 77, wherein the sequence read data is based on whole genome sequencing, targeted exome sequencing, or a combination thereof.
79. The method any of clauses 41 to 78, wherein the sequence read data is derived from a tissue biopsy sample, a liquid biopsy sample, or a combination thereof.
80. A method for diagnosing a disease, the method comprising: diagnosing that a subject has the disease based on a determination of the functional status for a variant based on a plurality of samples, wherein the functional status is determined according to the method of any one of clauses 41 to 79.
81. A method of selecting an anti-cancer therapy, the method comprising: responsive to determining a functional status for a variant based on a plurality of samples, selecting an anti-cancer therapy for the subject, wherein the functional status is determined according to the method of any one of clauses 41 to 79.
82. A method of treating a cancer in a subject, comprising: responsive to determining a functional status for a variant based on a plurality of samples, administering an effective amount of an anti-cancer therapy to the subject, wherein the functional status is determined according to the method of any one of clauses 41 to 79.
83. A method for monitoring cancer progression or recurrence in a subject, the method comprising:
   determining a presence of one or more first variants in a first sample obtained from the subject at a first time point,
   determining respective functional statuses of the one or more first variants, wherein the respective functional statuses are based on the functional status of the variant determined according to the method of any one of clauses 41 to 79;
   determining a presence of one or more second variants in a second sample obtained from the subject at a second time point;
   determining respective functional statuses of the one or more second variants, and
   comparing the one or more first variants and the one or more second variants in view of the respective functional statues, thereby monitoring the cancer progression or recurrence.
84. The method of clause 83, wherein the respective functional statuses for the second sample is determined according to the method of any one of clauses 41 to 79.
85. The method of clause 83 or clause 84, further comprising selecting an anti-cancer therapy for the subject in response to the cancer progression.
86. The method of clause 83 or clause 84, further comprising administering an anti-cancer therapy to the subject in response to the cancer progression.
87. The method of clause 83 or clause 84, further comprising adjusting an anti-cancer therapy for the subject in response to the cancer progression.
88. The method of any one of clauses 85 to 86, further comprising adjusting a dosage of the anti-cancer therapy or selecting a different anti-cancer therapy in response to the cancer progression.
89. The method of clause 88, further comprising administering the adjusted anti-cancer therapy to the subject.
90. The method of any one of clauses 84 to 89, wherein the first time point is before the subject has been administered an anti-cancer therapy, and wherein the second time point is after the subject has been administered the anti-cancer therapy.
91. The method of any one of clauses 84 to 90, wherein the subject has a cancer, is at risk of having a cancer, is being routine tested for cancer, or is suspected of having a cancer.
92. The method of any one of clauses 84 to 91, wherein the cancer is a solid tumor.
93. The method of any one of clauses 84 to 92, wherein the cancer is a hematological cancer.
94. The method of any one of clauses 84 to 93, wherein the anti-cancer therapy comprises chemotherapy, radiation therapy, immunotherapy, a targeted therapy, or surgery.
95. The method of any one of clauses 41 to 79, further comprising determining, identifying, or applying the functional status as a diagnostic metric associated with a sample of the plurality of samples.
96. The method of any one of clauses 41 to 79, further comprising generating a genomic profile for the subject based on the determination of the functional status.
97. The method of clause 96, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
98. The method of clause 96 or clause 97, wherein the genomic profile for the subject further comprises results from a nucleic acid sequencing-based test.
99. The method of any one of clauses 96 or clause 98, further comprising selecting an anti-cancer therapy, administering an anti-cancer therapy, or applying an anti-cancer therapy to the subject based on the generated genomic profile.
100. The method of any one of clauses 41 to 79, wherein the determination of the functional status of the variant based on a plurality of samples is used in making suggested treatment decisions for the subject.
101. The method of any one of clauses 41 to 79, wherein the determination of the functional status of the variant based on a plurality of samples for the sample is used in applying or administering a treatment to the subject.
102. A system comprising:
   one or more processors; and
   a memory communicatively coupled to the one or more processors and configured to store instructions that, when executed by the one or more processors, cause the system to perform a method comprising:
      receiving, at the one or more processors, sequence read data associated with a plurality of samples;
      determining, using the one or more processors, one or more feature attributes based on the sequence read data of the plurality of samples, the one or more feature attributes associated with one or more input features;
      categorizing, using the one or more processors, the plurality of samples into a plurality of input feature sub-types based on the one or more feature attributes, wherein an input feature of the one or more input features is associated with one or more input feature sub-types of the plurality of input feature sub-types;
      obtaining, using the one or more processors, one or more input feature values based on the plurality of input feature sub-types;
      inputting, using the one or more processors, the one or more input feature values into a statistical model; and
      determining, using the one or more processors, a functional status of a variant based on an output of the statistical model.
103. A non-transitory computer-readable storage medium storing one or more programs, the one or more programs comprising instructions, which when executed by one or more processors of a system, cause the system to:
   receive sequence read data associated with a plurality of samples;
   determine one or more feature attributes based on the sequence read data of the plurality of samples, the one or more feature attributes associated with one or more input features;
   categorize the plurality of samples into a plurality of input feature sub-types based on the one or more feature attributes, wherein an input feature of the one or more input features is associated with one or more input feature sub-types of the plurality of input feature sub-types;
   obtain one or more input feature values based on the plurality of input feature sub-types;
   input the one or more input feature values into a statistical model; and
   determine a functional status of a variant based on an output of the statistical model.
104. A method for determining an origin of a variant, the method comprising:
   receiving, using one or more processors, sequence read data associated with a plurality of samples;
   determining, using the one or more processors, one or more input feature values based on the sequence read data of the plurality of samples;
   inputting, using the one or more processors, the one or more input feature values into a statistical mod receiving, using one or more processors, sequence read data associated with a plurality of samples;
   determining, using the one or more processors, one or more feature attributes based on the sequence read data of the plurality of samples, the one or more feature attributes associated with one or more input features;
   categorizing, using the one or more processors, the one or more feature attributes into a plurality of input feature sub-types based on the one or more feature attributes, wherein an input feature of the one or more input features is associated with one or more input feature sub-types of the plurality of input feature sub-types;
   obtaining, using the one or more processors, one or more input feature values based on the plurality of input feature sub-types;
   inputting, using the one or more processors, the one or more input feature values into a statistical model; and
   determining, using the one or more processors, the origin of the variant based on an output of the statistical model.
105. The method of clause 104, wherein the origin of the variant comprises one of clonal hematopoiesis (CH) derived, tumor derived, or germline.
106. The method of any of clauses 104 to 105, wherein the plurality of input feature sub-types correspond to pre-determined sub-types.
107. The method of clause 106, wherein one or more of the pre-determined sub-types comprise quantiles based on the one or more input features.
108. The method of any of clauses 104 to 107, further comprising:
   inputting, using the one or more processors, the one or more feature attributes into a genomic database;
   determining, using the one or more processors, a gene co-mutation value indicative of a prevalence of gene co-mutations associated with the variant based on the genomic database; and
   inputting, using the one or more processors, the gene co-mutation value into the statistical model,
   wherein determining the origin of the variant is based on the gene co-mutation value.
109. The method of any of clauses 104 to 108, further comprising organizing a feature attribute of the one or more feature attributes into a first category based on a first input feature if a number of samples associated with the first input feature is below a pre-determined threshold.
110. The method of any of clauses 104 to 109, wherein the one or more input features are associated with one or more variant features, one or more sample features, one or more clinical features, or a combination thereof.
111. The method of clause 110, further comprising obtaining clinical information, wherein determining the one or more input features is further based on the clinical information.
112. The method of any of clauses 110 to 111, wherein the one or more variant features comprise a presence of a short variant, an absence of a short variant, a variant minor allele frequency, a germline status, a somatic status, a zygosity determination, a copy number alteration, a genomic rearrangement, or a combination thereof.
113. The method of any of clauses 110 to 112, wherein the one or more sample features comprise a bait-set, a tumor purity, a loss of heterozygosity (LOH) status, a LOH ploidy status, a LOH TP53 status, a LOH QC status, a microsatellite instability, a tumor mutational burden, a mutational signature, a gene co-mutation, or a combination thereof.
114. The method of any of clauses 110 to 113, wherein the one or more clinical features comprise an age of the individual, a sex of the individual, a disease ontology, a genomic ancestry of the individual, a bait set, or a combination thereof.
115. The method of any of clauses 104 to 114, wherein the output of the statistical model is indicative of the origin of the variant.
116. The method of any of clauses 104 to 115, wherein the output of the statistical model comprises one or more feature scores indicative of a relative importance of the one or more input features.
117. The method of any of clauses 104 to 116, wherein the statistical model is a trained machine learning model and trained by:
   receiving, using the one or more processors, training data including one or more training feature values based on one or more training feature sub-types associated with a plurality of training samples; and
   training, using the one or more processors, the machine learning model based on the training data.
118. The method of clause 117, wherein the one or more training feature sub-types are associated with one or more training features, the one or more training features comprising: one or more variant features, one or more sample features, one or more clinical features, or a combination thereof.
119. The method of any of clauses 117 to 118, further comprising obtaining training data, comprising:
   receiving, using one or more processors, training sequence read data associated with the plurality of training samples;
   determining, using the one or more processors, one or more training feature attributes based on the training sequence read data;
   organizing, using the one or more processors, the one or more training feature attributes into the one or more training feature sub-types;
   obtaining the one or more training feature values based on the one or more training feature sub-types;
   inputting, using the one or more processors, the one or more training feature values into an untrained statistical model;
   predicting, using the one or more processors, the origin of the variant based on the one or more training feature values;
   obtaining one or more training feature scores indicative of a relative importance of the one or more training features;
   updating one or more weights associated with a trained statistical model based on the one or more training feature scores.
120. The method of clause 119, further comprising organizing a training feature attribute of the one or more training feature attributes into a first category based on a first training feature, if a number of training samples of the plurality of training samples associated with the first training feature is below a pre-determined threshold.
121. The method of any of clauses 119 to 120, further comprising:
   inputting, using the one or more processors, the one or more training feature attributes into a genomic database;
   determining, using the one or more processors, a gene co-mutation value indicative of a number of gene co-mutations associated with the variant based on the genomic database; and
   inputting, using the one or more processors, the gene co-mutation value into the untrained statistical model,
   wherein predicting the origin of the variant is further based on the gene co-mutation value.
122. The method of any of clauses 119 to 121, further comprising:
   obtaining a pre-defined origin of the variant based on an orthogonal method;
   labeling the variant based on the pre-defined origin; and
   inputting the labeled pre-defined origin of the variant into the untrained statistical model,
   wherein updating one or more weights is based on the labeled pre-defined origin.
123. The method of clause 122, wherein the orthogonal method is based on variants identified in literature, variants identified in catalogue of somatic mutations in cancer (COSMIC), or a combination thereof.
124. The method of any of clauses 104 to 123, wherein the statistical model is a machine learning model.
125. The method of any of clauses 104 to 124, wherein the statistical model is part of a machine learning process.
126. The method of any of clauses 104 to 125, wherein the statistical model includes an artificial intelligence learning model.
127. The method of any of clauses 104 to 126, wherein the statistical model comprises a random forest model.
128. The method of any of clauses 104 to 127, wherein the statistical model comprises at least one of an extreme gradient boosting model, a logistic regression model, an elastic net model, a ridge regression model, a support vector machine model, a naïve-based model, a Gaussian naïve-based model, a limited expectation maximization model, a gradient boosting ensemble model, an adaboost model, a bagging model, a neural network model, a backpropagation model, a stochastic gradient descent model, a non-linear regression model, and a multivariate regression model.
129. The method any of clauses 104 to 128, further comprising reclassifying, using the one or more processors, the variant based on the origin of the variant.
130. The method any of clauses 104 to 129, further comprising generating, using the one or more processors, a report comprising the origin of the variant.
131. The method any of clauses 104 to 130, further comprising assigning, using the one or more processors, a therapy for an individual based on the origin of the variant.
132. The method any of clauses 104 to 131, further comprising determining, using the one or more processors, a treatment decision for an individual based on the origin of the variant.
133. The method any of clauses 104 to 132, wherein the method further comprises selecting an anti-cancer therapy to administer to the subject based on the determination of the origin of the variant.
134. The method any of clauses 104 to 133, further comprising administering, using the one or more processors, a treatment to an individual based on the origin of the variant.
135. The method any of clauses 104 to 134, wherein the method further comprises determining an effective amount of an anti-cancer therapy to administer to the subject based on the determination of the origin of the variant.
136. The method of clause 135, wherein the anti-cancer therapy comprises chemotherapy, radiation therapy, immunotherapy, a targeted therapy, or surgery.
137. The method any of clauses 104 to 136, further comprising monitoring, using the one or more processors, a prognosis of an individual based on the origin of the variant.
138. The method any of clauses 104 to 137, further comprising predicting, using the one or more processors, one or more clinical outcomes based on the origin of the variant.
139. The method any of clauses 104 to 138, wherein the sequence read data is based on whole genome sequencing, targeted exome sequencing, or a combination thereof.
140. The method any of clauses 104 to 139, wherein the sequence read data is derived from a tissue biopsy sample, a liquid biopsy sample, or a combination thereof.
141. A method for diagnosing a disease, the method comprising: diagnosing that a subject has the disease based on a determination of the origin for a variant based on a plurality of samples, wherein the origin is determined according to the method of any one of clauses 104 to 140.
142. A method of selecting an anti-cancer therapy, the method comprising: responsive to determining an origin for a variant based on a plurality of samples, selecting an anti-cancer therapy for the subject, wherein the origin is determined according to the method of any one of clauses 104 to 140.
143. A method of treating a cancer in a subject, comprising: responsive to determining an origin for a variant based on a plurality of samples, administering an effective amount of an anti-cancer therapy to the subject, wherein the origin is determined according to the method of any one of clauses 104 to 140.
144. A method for monitoring cancer progression or recurrence in a subject, the method comprising:
   determining a presence of one or more first variants in a first sample obtained from the subject at a first time point,
   determining respective origins of the one or more first variants, wherein the respective origins are based on the origin of the variant determined according to the method of any one of clauses 104 to 140;
   determining a presence of one or more second variants in a second sample obtained from the subject at a second time point;
   determining respective origins of the one or more second variants, and
   comparing the one or more first variants and the one or more second variants in view of the respective functional statues, thereby monitoring the cancer progression or recurrence.
145. The method of clause 144, wherein the respective origins for the second sample is determined according to the method of any one of clauses 104 to 140.
146. The method of clause 144 or clause 145, further comprising selecting an anti-cancer therapy for the subject in response to the cancer progression.
147. The method of clause 144 or clause 145, further comprising administering an anti-cancer therapy to the subject in response to the cancer progression.
148. The method of clause 144 or clause 145, further comprising adjusting an anti-cancer therapy for the subject in response to the cancer progression.
149. The method of any one of clauses 146 to 147, further comprising adjusting a dosage of the anti-cancer therapy or selecting a different anti-cancer therapy in response to the cancer progression.
150. The method of clause 149, further comprising administering the adjusted anti-cancer therapy to the subject.
151. The method of any one of clauses 145 to 150, wherein the first time point is before the subject has been administered an anti-cancer therapy, and wherein the second time point is after the subject has been administered the anti-cancer therapy.
152. The method of any one of clauses 145 to 151, wherein the subject has a cancer, is at risk of having a cancer, is being routine tested for cancer, or is suspected of having a cancer.
153. The method of any one of clauses 145 to 152, wherein the cancer is a solid tumor.
154. The method of any one of clauses 145 to 153, wherein the cancer is a hematological cancer.
155. The method of any one of clauses 145 to 154 wherein the anti-cancer therapy comprises chemotherapy, radiation therapy, immunotherapy, a targeted therapy, or surgery.
156. The method of any one of clauses 104 to 140, further comprising determining, identifying, or applying the origin as a diagnostic metric associated with a sample of the plurality of samples.
157. The method of any one of clauses 104 to 140, further comprising generating a genomic profile for the subject based on the determination of the origin.
158. The method of clause 157, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
159. The method of clause 157 or clause 158, wherein the genomic profile for the subject further comprises results from a nucleic acid sequencing-based test.
160. The method of any one of clauses 157 to 159, further comprising selecting an anti-cancer therapy, administering an anti-cancer therapy, or applying an anti-cancer therapy to the subject based on the generated genomic profile.
161. The method of any one of clauses 104 to 140, wherein the determination of the origin of the variant based on a plurality of samples is used in making suggested treatment decisions for the subject.
162. The method of any one of clauses 104 to 140, wherein the determination of the origin of the variant based on a plurality of samples for the sample is used in applying or administering a treatment to the subject.
163. A system comprising:
   one or more processors; and
   a memory communicatively coupled to the one or more processors and configured to store instructions that, when executed by the one or more processors, cause the system to perform a method comprising:
      receiving, at the one or more processors, sequence read data associated with a plurality of samples;
      determining, using the one or more processors, one or more feature attributes based on the sequence read data of the plurality of samples, the one or more feature attributes associated with one or more input features;
      categorizing, using the one or more processors, the plurality of samples into a plurality of input feature sub-types based on the one or more feature attributes, wherein an input feature of the one or more input features is associated with one or more input feature sub-types of the plurality of input feature sub-types;
      obtaining, using the one or more processors, one or more input feature values based on the plurality of input feature sub-types;
      inputting, using the one or more processors, the one or more input feature values into a statistical model; and
      determining, using the one or more processors, an origin of a variant based on an output of the statistical model.
164. A non-transitory computer-readable storage medium storing one or more programs, the one or more programs comprising instructions, which when executed by one or more processors of a system, cause the system to:
   receive sequence read data associated with a plurality of samples;
   determine one or more feature attributes based on the sequence read data of the plurality of samples, the one or more feature attributes associated with one or more input features;
   categorize the plurality of samples into a plurality of input feature sub-types based on the one or more feature attributes, wherein an input feature of the one or more input features is associated with one or more input feature sub-types of the plurality of input feature sub-types;
   obtain one or more input feature values based on the plurality of input feature sub-types;
   input the one or more input feature values into a statistical model; and
   determine an origin of a variant based on an output of the statistical model.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for determining a functional status of a variant, the method comprising:
providing a plurality of nucleic acid molecules obtained from a plurality of samples from a plurality of subjects;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, using one or more processors, sequence read data associated with the plurality of samples;
determining, using the one or more processors, one or more feature attributes based on the sequence read data of the plurality of samples, the one or more feature attributes associated with one or more input features;
categorizing, using the one or more processors, the plurality of samples into a plurality of input feature sub-types based on the one or more feature attributes, wherein an input feature of the one or more input features is associated with one or more input feature sub-types of the plurality of input feature sub-types;
obtaining, using the one or more processors, one or more input feature values based on the plurality of input feature sub-types;
inputting, using the one or more processors, the one or more input feature values into a statistical model; and
determining, using the one or more processors, the functional status of the variant based on an output of the statistical model,
wherein the functional status is indicative of a level of pathogenicity of the variant.

2. The method of claim 1, wherein the functional status comprises a known pathogenic status of the variant or a non-known pathogenic status of the variant.

3. The method of claim 2, wherein the non-known pathogenic status comprises a likely pathogenic status of the variant, a variant of unknown significance status of the variant, a benign status of the variant, or a combination thereof.

4. The method of claim 2, wherein the non-known pathogenic status comprises a variant of unknown significance status of the variant, a benign status of the variant, or a combination thereof.

5. The method of claim 1, wherein the plurality of input feature sub-types correspond to pre-determined sub-types.

6. The method of claim 5, wherein one or more of the pre-determined sub-types comprise quantiles based on the one or more input features.

7. The method of claim 1, further comprising:
inputting, using the one or more processors, the one or more feature attributes into a genomic database;
determining, using the one or more processors, a gene co-mutation value indicative of a prevalence of gene co-mutations associated with the variant based on the genomic database; and
inputting, using the one or more processors, the gene co-mutation value into the statistical model,
wherein determining the functional status is based on the gene co-mutation value.

8. The method of claim 1, further comprising organizing a feature attribute of the one or more feature attributes into a first category based on a first input feature if a number of samples associated with the first input feature is below a pre-determined threshold.

9. The method of claim 1, wherein the one or more input features are associated with one or more variant features, one or more sample features, one or more clinical features, or a combination thereof.

10. The method of claim 9, wherein the one or more variant features comprise a presence of a short variant, an absence of a short variant, a variant minor allele frequency, a germline status, a somatic status, a zygosity determination, a copy number alteration, a genomic rearrangement, or a combination thereof.

11. The method of claim 9, wherein the one or more sample features comprise a bait-set, a tumor purity, a loss of heterozygosity (LOH) status, a LOH ploidy status, a LOH TP53 status, a LOH QC status, a microsatellite instability, a tumor mutational burden, a mutational signature, a gene co-mutation, or a combination thereof.

12. The method of claim 9, wherein the one or more clinical features comprise an age of the individual, a sex of the individual, a disease ontology, a genomic ancestry of the individual, a bait set, or a combination thereof.

13. The method of claim 1, wherein the output of the statistical model is indicative of a functional status of the variant.

14. The method of claim 1, wherein the statistical model is a trained machine learning model and trained by:
receiving, using the one or more processors, training data including one or more training feature values based on one or more training feature sub-types associated with a plurality of training samples; and
training, using the one or more processors, the machine learning model based on the training data.

15. The method of claim 14, wherein the one or more training feature sub-types are associated with one or more training features, the one or more training features comprising: one or more variant features, one or more sample features, one or more clinical features, or a combination thereof.

16. The method of claim 14, further comprising obtaining training data, comprising:
receiving, using one or more processors, training sequence read data associated with the plurality of training samples;
determining, using the one or more processors, one or more training feature attributes based on the training sequence read data;
organizing, using the one or more processors, the one or more training feature attributes into the one or more training feature sub-types;
obtaining the one or more training feature values based on the one or more training feature sub-types;
inputting, using the one or more processors, the one or more training feature values into an untrained statistical model;
predicting, using the one or more processors, the functional status of the variant based on the one or more training feature values;
obtaining one or more training feature scores indicative of a relative importance of the one or more training features; and
updating one or more weights associated with a trained statistical model based on the one or more training feature scores.

17. The method of claim 16, further comprising organizing a training feature attribute of the one or more training feature attributes into a first category based on a first training feature, if a number of training samples of the plurality of training samples associated with the first training feature is below a pre-determined threshold.

18. The method of claim 16, further comprising:
inputting, using the one or more processors, the one or more training feature attributes into a genomic database;
determining, using the one or more processors, a gene co-mutation value indicative of a number of gene co-mutations associated with the variant based on the genomic database; and
inputting, using the one or more processors, the gene co-mutation value into the untrained statistical model,
wherein predicting the functional status is further based on the gene co-mutation value.

19. The method of claim 16, further comprising:
obtaining a pre-defined functional status of the variant based on an orthogonal method;
labeling the variant based on the pre-defined functional status; and
inputting the labeled pre-defined functional status of the variant into the untrained statistical model,
wherein updating one or more weights is based on the labeled pre-defined functional status.

20. A system comprising:
one or more processors; and
a memory communicatively coupled to the one or more processors and configured to store instructions that, when executed by the one or more processors, cause the system to perform a method comprising:
receiving, at the one or more processors, sequence read data associated with a plurality of samples;
determining, using the one or more processors, one or more feature attributes based on the sequence read data of the plurality of samples, the one or more feature attributes associated with one or more input features;
categorizing, using the one or more processors, the plurality of samples into a plurality of input feature sub-types based on the one or more feature attributes, wherein an input feature of the one or more input features is associated with one or more input feature sub-types of the plurality of input feature sub-types;
obtaining, using the one or more processors, one or more input feature values based on the plurality of input feature sub-types;
inputting, using the one or more processors, the one or more input feature values into a statistical model; and
determining, using the one or more processors, a functional status of a variant based on an output of the statistical model.

21. A non-transitory computer-readable storage medium storing one or more programs, the one or more programs comprising instructions, which when executed by one or more processors of a system, cause the system to:
receive sequence read data associated with a plurality of samples;
determine one or more feature attributes based on the sequence read data of the plurality of samples, the one or more feature attributes associated with one or more input features;
categorize the plurality of samples into a plurality of input feature sub-types based on the one or more feature attributes, wherein an input feature of the one or more input features is associated with one or more input feature sub-types of the plurality of input feature sub-types;
obtain one or more input feature values based on the plurality of input feature sub-types;
input the one or more input feature values into a statistical model; and
determine a functional status of a variant based on an output of the statistical model.
